(19) United States
(12) Patent Application Publication
(10) Pub. No.: US 2026/0263437 A1
(43) Pub. Date: Sep. 10, 2026

(54) HYDROGEL COMPOSITIONS FOR USE FOR DEPLETION OF TUMOR ASSOCIATED MACROPHAGES

(71) Applicant: Wisconsin Alumni Research Foundation, Madison, WI (US)

(72) Inventors: Quanyin Hu, Waunakee, WI (US); Yingyue Ding, Madison, WI (US); Zhaoting Li, Madison, WI (US)

(73) Assignee: Wisconsin Alumni Research Foundation, Madison, WI (US)

(21) Appl. No.: 18/722,697

(22) PCT Filed: Dec. 21, 2022
(86) PCT No.: PCT/US2022/053597

§ 371 Date: Jun. 21, 2024

Related U.S. Application Data
(60) Provisional application No. 63/294,247, filed on Dec. 28, 2021.

(51) Int. Cl. A61K 31/444; A61K 9/51; A61K 47/36; A61K 47/68; A61K 47/69; A61P 35/00; C07K 16/28

(52) U.S. Cl. CPC A61K 31/444; A61K 9/5161; A61K 47/36; A61K 47/6849; A61K 47/6901; A61P 35/00; C07K 16/2818

(57) ABSTRACT

A depot pharmaceutical formulation comprises a biocompatible hydrogel, the hydrogel encapsulating an effective amount of a colony-stimulating factor receptor (CSFR) inhibitor, wherein the CSFR inhibitor blocks CSF receptors on tumor associated macrophages (TAMs); and an effective amount of a platelet-conjugated anti-immune checkpoint inhibitor (ICI) antibody, wherein the platelet-conjugated anti-ICI antibody activates T-cells.

### CROSS-REFERENCE TO RELATED APPLICATIONS

This application claims priority to U.S. Provisional Application 63/294,247 filed on Dec. 28, 2021, which is incorporated herein by reference in its entirety.

### STATEMENT REGARDING FEDERALLY SPONSORED RESEARCH & DEVELOPMENT

This invention was made with government support under CA014520 awarded by the National Institutes of Health. The government has certain rights in the invention.

### FIELD OF THE DISCLOSURE

The present disclosure is related to depot pharmaceutical formulations, methods of depleting tumor associated macrophages in a tumor microenvironment, and methods of treating a post-surgical tumor site.

### BACKGROUND

Surgery remains the foremost treatment option for patients with solid tumors, however, tumor recurrence frequently occurs, leading to a low rate of long-term survival. Cancer immunotherapy, especially immune checkpoint blockade, has been demonstrated as one of the most potent anti-cancer recurrence strategies either as monotherapy or in combination with other treatment modalities. Specifically, by blocking the pathway of programmed cell death protein 1 (PD-1) and programmed cell death ligand 1 (PDL-1) to regain suppressed anti-tumor immunity, promising therapeutic efficacy in treating cancer recurrence has been observed in clinical applications. However, intricate physiological environmental changes after surgery, especially wound healing-triggered inflammatory conditions and inflammation-responsive immunosuppression, could diminish the efficacy of cancer immunotherapy, leading to the low objective response rate in the clinic. Furthermore, immune-related adverse events due to over-activated T cells by immune checkpoint blockade remain a concern.

What is needed are novel compositions and methods for the local treatment of post-surgical tumor recurrence.

### BRIEF SUMMARY

In one aspect, a depot pharmaceutical formulation comprises a biocompatible hydrogel, the hydrogel encapsulating an effective amount of a colony-stimulating factor receptor (CSFR) inhibitor, wherein the CSFR inhibitor blocks CSF receptors on tumor associated macrophages (TAMs); and an effective amount of a platelet-conjugated anti-immune checkpoint inhibitor (ICI) antibody, wherein the platelet-conjugated anti-ICI antibody activates T-cells.

In another aspect, a method of treating a post-surgical tumor site in a subject comprises depositing at the post-surgical tumor site an effective amount of the depot formulation described above.

In a further aspect, a method of treating a post-surgical tumor site in a subject comprises depositing at the post-surgical tumor site an effective amount of a depot pharmaceutical formulation comprising a biocompatible hydrogel, the hydrogel encapsulating an effective amount of a colony-stimulating factor receptor (CSFR) inhibitor, wherein the CSFR inhibitor blocks CSF receptors on tumor associated macrophages (TAMs); and systemically administering a systemic formulation comprising a platelet-conjugated anti-immune checkpoint inhibitor (ICI) antibody, wherein the platelet-conjugated anti-ICI antibody activates T-cells.

### BRIEF DESCRIPTION OF THE DRAWINGS

FIGS. 1A-I show the preparation and characterization of PLX-NP-P-aPD-1@Gel. FIG. 1A is a schematic illustration of the mechanism of tumor immune suppressive microenvironment modulation capability of PLX-NP and P-aPD-1 loaded alginate-based hydrogel in the tumor recurrence model. MHC, major histocompatibility complex; TCR, T-cell receptor. FIG. 1B shows the release profile of PLX-NP in vitro at a pH of 6.5. Data are presented as mean ±s.d. (n=3). FIG. 1C shows confocal microscopy images of anti-PD-1 conjugated platelets. Scale bar, 20 μm. Green: FITC-labeled aPD-1; Red: WGA 594-labeled platelet. FIG. 1D shows confocal microscopy images of NP and P-aPD-1 in the hydrogel. Scale bar, 50 μm. Green: FITC-labeled NP; Red: Rhodamine B-labeled P-aPD-1. FIG. 1E is a cryo-SEM image of PLX-NP and P-aPD-1 co-loaded hydrogel. (Scale bar=2 μm, red arrow: platelets, green arrow: PLX-NP). FIG. 1F-1G are graphs showing the in vitro release profiles of platelets (1F) and aPD-1 (1G) from the hydrogel. Data are presented as mean ±s.d. (n=3). FIG. 1H-I show the degradation profiles of Cy5-alginate hydrogel in vivo represented by radiant efficiency (FIG. 1H) and IVIS spectrum imaging (FIG. 1I). Data are presented as mean ±s.d. (n=3).

FIGS. 2A-G show the results of the evaluation of in vivo TAMs depletion capability and T cell infiltration of PLX-NP@Gel. FIGS. 2A-B are representative flow cytometry plots of TAMs (2A) and CD8+T cells (2B) in the recurrent tumor tissues after treatments with saline, NP@Gel, PLX, PLX-NP, and PLX-NP@Gel. FIGS. 2C-E show the results of quantitative analysis of intratumoral densities of F4/80⁺macrophages (2C), CD8⁺ T cells (2D), and IFNγ⁺CD8⁺T cells (2E) in the recurrent tumor tissues after treatments with saline, NP@Gel, PLX, PLX-NP, and PLX-NP@Gel. Data are presented as mean ±s.d. (n=5), *P<0.05, **P<0.01, ***P<0.001, one-way ANOVA followed by multiple comparisons test. FIGS. 2F-G are representative confocal microscopy images of immune-stained TAMs (2F) and CD8⁺ T cells (2G) in the saline group and PLX-NP@Gel group. Scale bar, 100 μm.

FIGS. 3A-F show the results of evaluation of in vivo treatment efficacy of PLX-NP-P-aPD-1@Gel in CT26 and B16F10 tumor recurrence model. FIG. 3A is graphs showing region-of-interest analysis of bioluminescence intensity of the tumors among different treatment groups in CT26 tumor recurrence model (n=6). Saline; NP-P@Gel, blank nanoparticle and unmodified platelets co-loaded hydrogel; PLX-aPD-1@Gel, free PLX and aPD-1 co-loaded hydrogel; PLX-NP@Gel, PLX-NP loaded hydrogel; P-aPD-1@Gel, P-aPD-1 loaded hydrogel; PLX-NP+P-aPD-1, free PLX-NP and P-aPD-1; PLX-NP-P-aPD-1@Gel, PLX-NP and P-aPD-1 co-loaded hydrogel. The doses of PLX and aPD-1 were 5 mg/kg and 0.1 mg/kg, respectively. FIG. 3B shows the survival curves of mice treated with different treatment groups in CT26 tumor recurrence model (n=6). Data were analyzed with Log-rank (Mantel-Cox) test, PLX-NP-P-aPD-1@Gel vs PLX-NP+P-aPD-1, **P<0.01. FIG. 3C is a graph showing the weight change of mice among different treatment groups in CT26 tumor recurrence model. Data are presented as mean ±s. d. (n=6). FIG. 3D is graphs showing region-of-interest analysis of bioluminescence intensity of the tumors among different treatment groups in B16F10 tumor recurrence model (n=6). FIG. 3E shows the survival curves of mice treated with different treatment groups in B16F10 tumor recurrence model (n=6). Data were analyzed with Log-rank (Mantel-Cox) test, PLX-NP-P-aPD-1@Gel vs PLX-NP+P-aPD-1, *P<0.05. FIG. 3F is a graph showing the weight change of mice among different treatment groups in B16F10 tumor recurrence model. Data are presented as mean ±s. d. (n=6).

FIGS. 4A-G show the results of evaluation of in vivo immune response of PLX-NP-P-aPD-1@Gel in B16F10 tumor recurrence model. FIG. 4A is a representative confocal microscopy image of platelets and the formation of platelet-derived microparticles at the tumor sites. Scale bar, 100 μm. FIGS. 4B-E are graphs showing the quantitative analysis of the flow cytometry populations of F4/80⁺ macrophages (4B), CD3⁺ T cells (4C), CD8⁺ T cells (4D), and Granzyme B⁺ CD8⁺ T cells (4E) in the recurrent tumor tissues after treatments with saline, NP-P@Gel, PLX-aPD-1@Gel, PLX-NP@Gel, P-aPD-1@Gel, PLX-NP-P-aPD-1, and PLX-NP-P-aPD-1@Gel. Data are presented as mean ±s.d. (n=4). *P<0.05, **P<0.01, ***P<0.001, ns: no significant difference, one-way ANOVA followed by multiple comparisons test. f, IFNγ levels in the tumors detected by enzyme-linked immunosorbent assay (ELISA) after different treatments. Data are presented as mean ±s.d. (n=4). *P<0.05, **P<0.01, one-way ANOVA followed by multiple comparisons test. FIG. 4E shows the IFNγ cytokine level was elevated in the PLX-NP+P-aPD-1@Gel group FIG. 4G are representative confocal microscopy images of immune-stained CD8⁺ T cells and TAMs in the saline group and PLX-NP-P-aPD-1@Gel group. Scale bar, 100 μm.

FIGS. 5A-E show the results of evaluation of in vivo treatment efficacy of PLX-NP-P-aPD-1@Gel in metastatic 4T1 breast tumor recurrence model. FIG. 5A shows representative bioluminescence images of tumor-bearing mice following different treatments on day 0, day 7, day 14 and day 21. The dose of PLX and aPD-1 was 5 mg/kg and 0.1 mg/kg, respectively. FIG. 5B is a graph showing data analysis of bioluminescence intensity of the tumors among different treatment groups. (n=6). ***P<0.001, two-way ANOVA followed by multiple comparisons test. FIG. 5C shows survival curves of the mice treated with different treatment groups. (n=6). Data were analyzed with Log-rank (Mantel-Cox) test, PLX-NP-P-aPD-1@Gel vs PLX-NP+P-aPD-1, **P<0.01. FIG. 5D is a graph showing data analysis of the number of metastatic nodules on the surface of the lungs on day 21 (n=3). *P<0.05, one-way ANOVA followed by multiple comparisons test. FIG. 5E shows representative images of the lungs and H&E assay after different treatments on day 21.

FIGS. 6A-G show the results of evaluation of in vivo treatment efficacy of PLX-NP-P-aPD-1@Gel in different tumor recurrence models including the sarcoma S180 tumor model, B16F10 melanoma model in T cell-deficient mice, and distant tumor model. FIG. 6A is the tumor growth curves among different treatment groups in sarcoma S180 tumor recurrence model (n=6). Saline; NP-P@Gel, blank nanoparticle and unmodified platelets co-loaded hydrogel; PLX-aPD-1@Gel, free PLX and aPD-1 co-loaded hydrogel; PLX-NP@Gel, PLX-NP loaded hydrogel; P-aPD-1@Gel, P-aPD-1 loaded hydrogel; PLX-NP+P-aPD-1, free PLX-NP and P-aPD-1; PLX-NP-P-aPD-1@Gel, PLX-NP and P-aPD-1 co-loaded hydrogel. The doses of PLX and aPD-1 were 5 mg/kg and 0.1 mg/kg, respectively. FIG. 6B is representative tumor images after different treatment on day 21 (scale bar=1 cm). FIG. 6C shows the survival curves of the mice with different treatments in S180 tumor recurrence model (n=6). Data were analyzed with Log-rank (Mantel-Cox) test, PLX-NP-P-aPD-1@Gel vs PLX-NP+P-aPD-1, **P<0.01. FIG. 6D is representative bioluminescence images of T cell-deficient B16F10 tumor-bearing mice following different treatments on day 0, day 7, day 14, day 21 (n=5). FIG. 6E shows the survival curves of the mice treated with different treatment groups in B16F10 tumor recurrence model in T cell-deficient mice. FIG. 6F is a schematic illustration of the establishment and treatment strategy of the distant tumor model. FIG. 6G is a graph showing the tumor growth curve of the distant tumor after different treatments. ***P<0.001, two-way ANOVA followed by multiple comparisons test.

FIGS. 7A-I show the results of evaluation of in vivo treatment efficacy of local implantation of PLX-NP@Gel with intravenous injection of P-aPD-1 in B16F10 tumor recurrence model. FIG. 7A is a schematic illustration of the experimental design of local implantation of PLX-NP@Gel with intravenous injection of P-aPD-1 in B16F10 tumor recurrence model. FIG. 7B shows the survival curves of the mice treated with different groups. (n=6). Saline; PLX-NP@Gel; PLX-NP@Gel+aPD-1, PLX-NP@Gel and intravenous injection of free aPD-1; PLX-NP@Gel+P-aPD-1, PLX-NP@Gel and intravenous injection of P-aPD-1. The dose of PLX was 5 mg/kg, and the dose of aPD-1 was 0.5 mg/kg. **P<0.001, log-rank test, PLX-NP@Gel+P-aPD-1 vs PLX-NP@Gel+aPD-1. FIG. 7C is graphs showing the tumor volume change of the mice treated with different groups in three weeks. (n=6). FIG. 7D shows representative tumor images of mice among different groups at day 21. Scale bar, 1 cm. FIG. 7E shows the tumor weight of mice among different groups at day 21. Data are presented as mean ±s.d. (n=5). *P<0.05, one-way ANOVA followed by multiple comparisons test. FIGS. 7F-G are the quantitative analysis of the flow cytometry results of CD8⁺ T cells (7F) and Granzyme B⁺CD8⁺ T cells (7G) in the recurrent tumor tissues after different treatments. Data are presented as mean ±s.d. (n=4). **P<0.01, ***P<0.001, one-way ANOVA followed by multiple comparisons test. FIGS. 7H-I are graphs showing the IFNγ (7H) and TNFα (7I) levels within the tumor as detected by ELISA one week after the treatment among different groups. Data are presented as mean ±s.d. (n=4). *P<0.05, **P<0.01, ***P<0.001, one-way ANOVA followed by multiple comparisons test.

FIGS. 8A-B show the average size and TEM image of PLX-NP. FIG. 8A is a graph showing the average size of PLX-NP measured by DLS. FIG. 8B is a TEM image of PLX-NP; Scale bar, 200 nm.

FIGS. 9A-B are graphs of the cell viability of RAW264.7 cell line (9A) and NIH/3T3 cell line (9B) treated with PLX-NP. Cells were treated with various concentrations of PLX-NP ranging from 0-125 μg/ml for 24 hours (n=3).

FIG. 10 is fluorescent confocal microscopy images of the collagen binding evaluation of free platelets and P-aPD-1. The confocal dish without pre-treatment of collagen was set as blank control. Platelets and P-aPD-1 were labeled with NHS-Rhodamine B for confocal microscopy. Scale bar, 50 μm.

FIG. 11 shows flow cytometry histograms of surface protein expression of platelets and P-aPD-1 (n=3). Anti-mouse CD8 antibody was selected as the isotype control antibody and CD62P was the platelet activation marker.

FIGS. 12A-B shows the morphologies of alginate-based hydrogel before (12A) and after (12B) adding Ca²⁺.

FIG. 13 shows the loading efficiency of PLX-NP and platelets in the alginate hydrogel.

FIG. 14 is representative cryo-SEM images of the PLX-NP and P-aPD-1 co-loaded hydrogel at different magnifications.

FIG. 15 is a graph showing the release profile of PLX-NP@Gel in vivo.

FIG. 16 shows the gating strategy for flow cytometry analysis of IFNγ.

FIG. 17 shows the results of a representative flow cytometry assay for IFNγ staining.

FIG. 18 shows representative bioluminescence images of CT26-Luc tumor-bearing mice following different treatments on day 0, day 7, day 14, and day 21. Saline; NP-P@Gel, blank nanoparticle and unmodified platelets co-loaded hydrogel; PLX-aPD-1@Gel, free PLX and aPD-1 co-loaded hydrogel; PLX-NP@Gel, PLX-NP loaded hydrogel; P-aPD-1@Gel, P-aPD-1 loaded hydrogel; PLX-NP+P-aPD-1, free PLX-NP and P-aPD-1; PLX-NP-P-aPD-1@Gel, PLX-NP and P-aPD-1 co-loaded hydrogel. The doses of PLX and aPD-1 were 5 mg/kg and 0.1 mg/kg, respectively.

FIG. 19 shows representative bioluminescence images of B16F10-Luc tumor-bearing mice following different treatments on day 0, day 7, day 14, and day 21. Saline; NP-P@Gel, blank nanoparticle and unmodified platelets co-loaded hydrogel; PLX-aPD-1@Gel, free PLX and aPD-1 co-loaded hydrogel; PLX-NP@Gel, PLX-NP loaded hydrogel; P-aPD-1@Gel, P-aPD-1 loaded hydrogel; PLX-NP+P-aPD-1, free PLX-NP and P-aPD-1; PLX-NP-P-aPD-1@Gel, PLX-NP and P-aPD-1 co-loaded hydrogel. The doses of PLX and aPD-1 were 5 mg/kg and 0.1 mg/kg, respectively.

FIG. 20 shows representative H&E staining images of the heart, liver, spleen, lung, and kidney in saline and PLX-NP-P-aPD-1@Gel treatment groups (n=3). Scale bar is 100 μm.

FIG. 21 is a tumor growth curve after different treatments. The dose of PLX was 5 mg/kg, the dose of aPD-1 for the PLX-NP-P-aPD-1@Gel was 0.1 mg/kg, and the dose of aPD-1 for the PLX-NP@Gel+P-aPD-1 was 0.5 mg/kg. **P<0.001, ***P<0.001, two-way ANOVA followed by multiple comparisons test.

The above-described and other features will be appreciated and understood by those skilled in the art from the following detailed description, drawings, and appended claims.

### DETAILED DESCRIPTION

One of the mechanisms that limit immune checkpoint blockade therapy in recurrent cancer is the hindered T cell migration towards and within a tumor site due to the tumor immune suppressive microenvironment, resulting in low availability of tumor infiltrating lymphocytes and progressive tumor growth. Studies have shown that immune suppressive cells, especially tumor associated macrophages (TAMs), are highly implicated in suppressing anti-tumor immune functions of T cells and directly facilitating tumor cell immune escape. Specifically, a recent study has identified that CD8+ T cell accumulation and infiltration at a tumor site have been primarily enhanced by depleting TAMs, resulting in improved immunotherapy efficacy. Pexidartinib (PLX) is an FDA-approved small molecule drug that shows the remarkable selectivity to block the CSF1 receptors on TAMs with negligible cytotoxicity against normal cells, providing a potential target for TAM depletion for modulating tumor immunosuppressive microenvironment and enhancing immunotherapy by improving T cell infiltration. Thus, the combination of TAMs depletion and immune checkpoint blockade could be critical for augmenting anti-tumor immunotherapy outcomes.

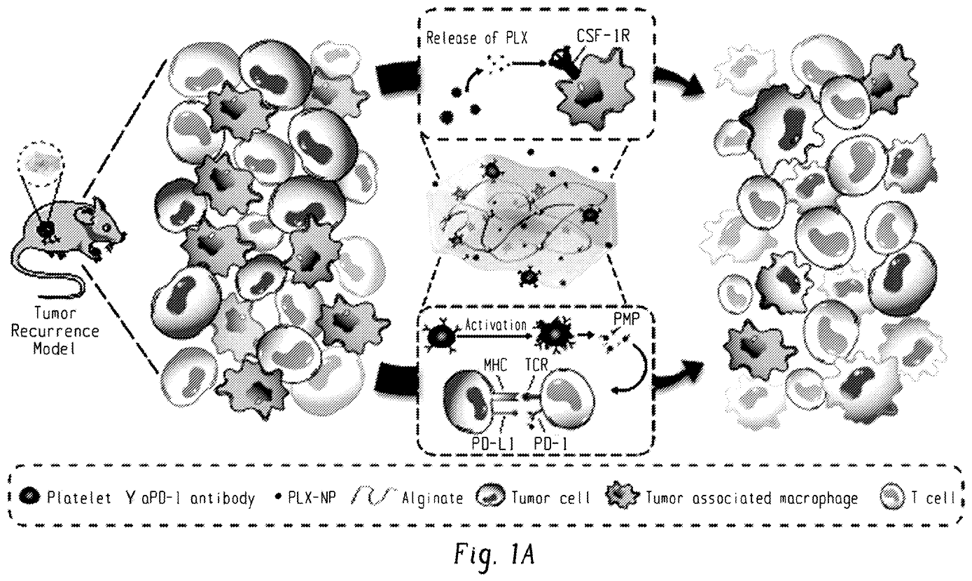

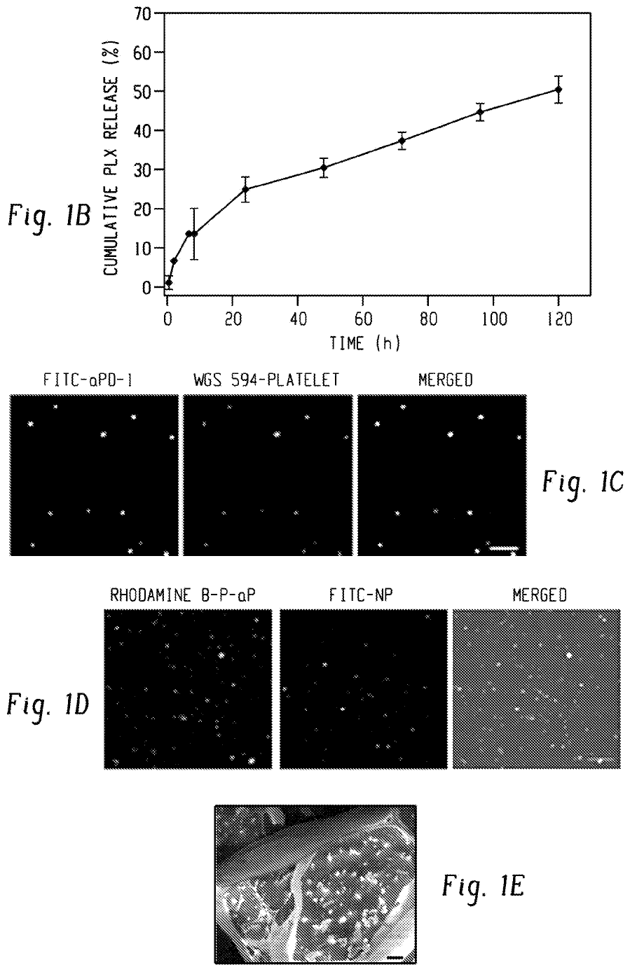

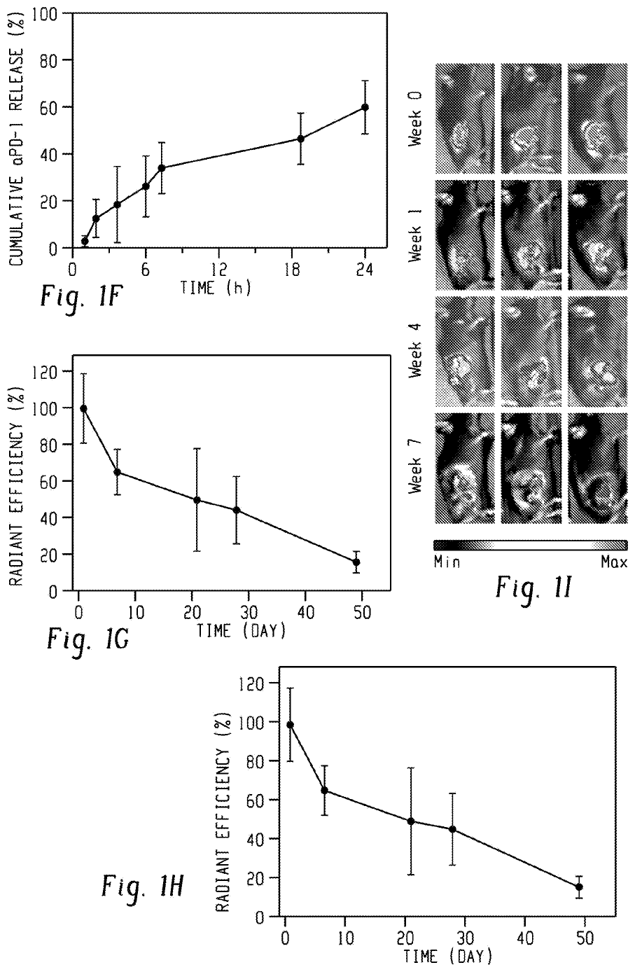

Described herein is a biocompatible alginate-based hydrogel encapsulating PLX-loaded nanoparticles (designated PLX-NP) and anti-PD-1-conjugated platelets (designated P-aPD-1) for post-surgery tumor recurrence treatment by locally and sustainedly releasing PLX and anti-PD-1 antibodies for depleting TAMs and activating infiltrated T cells, respectively (FIG. 1A). A biodegradable dextran nanoparticle was formulated to encapsulate and bioresponsively release PLX to block CSF1 receptors to eliminate TAMs in the tumor microenvironment. Anti-PD-1 antibodies were conjugated on the surface of platelets, where the inflammation environment secondary to the surgical expose could activate platelet to release anti-PD-1 antibodies to block PD-1 receptors on infiltrated T cells in the format of platelet-derived microparticles (PMPs). Both PLX-NP and P-aPD-1 were harbored in the hydrogel to form a local delivery reservoir, in which PLX was gradually released to deplete TAMs to recruit T cells toward tumor parenchyma, favoring the subsequent anti-PD-1 immunotherapy. To further extend the application of this combination immunotherapy strategy, P-aPD-1 was systemically administered to synergize with local PLX-NP loaded hydrogel to promote sustained immune response against tumor recurrence, where less than 20% of patients are benefiting from a systemic injection of checkpoint inhibitors clinically. Without being held to theory, it is believed that this hydrogel-based delivery system could enhance post-surgery tumor recurrence treatment by gradually releasing PLX to deplete TAMs, facilitating T cell recruitment and infiltration, promoting local and systemic platelet-mediated immune checkpoint inhibitors delivery.

In an aspect, a depot formulation comprises a biocompatible hydrogel, the biocompatible hydrogel encapsulating an effective amount of a colony-stimulating factor receptor (CSFR) inhibitor, wherein the CSFR inhibitor blocks CSF receptors on tumor associated macrophages (TAMs); and an effective amount of a platelet-conjugated anti-immune checkpoint inhibitor (ICI) antibody, wherein the platelet-conjugated anti-ICI antibody activates T-cells.

The depot formulation comprises a biocompatible hydrogel, such as an alginate hydrogel. “Hydrogel” refers to a substance formed when an organic polymer (natural or synthetic) is cross-linked via covalent, ionic, or hydrogen bonds to create a three-dimensional open-lattice structure that entraps water molecules to form a gel. “Biocompatible hydrogel refers” to a hydrogel that is not toxic to living cells. “Alginate” is a collective term used to refer to linear polysaccharides formed from beta-D-mannuronate and alpha-L-guluronate in any M/G ratio, as well as salts and derivatives thereof.

Examples of materials that can be used to form a biocompatible hydrogel include polysaccharides such as alginate, polyphosphazines, poly(acrylic acids), poly(methacrylic acids), poly(alkylene oxides), poly(vinyl acetate), polyvinylpyrrolidone (PVP), and copolymers and blends. Additional materials for forming hydrogels include agarose, chitosan, dextran, dextran sulfate, heparan, heparan sulfate, cellulose sulphate, carrageenan, gellan gum, xanthan gum, guar gum, chondroitin sulfate, hyaluronic acid, collagen, gelatin, poly(N-isopropyl acrylamide). Combinations of the foregoing materials may be employed.

The biocompatible hydrogel encapsulates an effective amount of a CSFR inhibitor, wherein the CSFR inhibitor blocks CSF receptors on tumor associated macrophages (TAMs). In an aspect, the CSFR inhibitor is encapsulated in a sustained release nanoparticle, specifically a biodegradable sustained release nanoparticle, which is then encapsulated in the biocompatible hydrogel.

As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain aspects, components generated by the breakdown of a biodegradable material do not induce inflammation and/or other adverse effects in vivo. Biodegradable materials may be enzymatically broken down, or broken down by hydrolysis, for example, into their component polymers. Breakdown of biodegradable materials (including, for example, biodegradable polymeric materials) may include hydrolysis of ester bonds, cleavage of urethane linkages, and the like.

Exemplary materials for biodegradable nanoparticles include poly-lactic acid (PLA); poly-D-L-glycolide (PLG); poly-D-L-lactide-co-glycolide (PLGA), poly-alkyl-cyanoacrylate (PCA), poly-8-caprolactone, gelatin, alginate, chitosan, agarose, polysaccharides, and proteins. Biodegradable nanoparticles can be made by techniques known in the art such as solvent evaporation, spontaneous emulsification, nanoprecipitation, salting out, polymerization, or ionic gelation of hydrophilic polymers, for example.

In an aspect, the biodegradable nanoparticle comprises a polysaccharide. The term “polysaccharide” refers to a polymer of sugars. Polysaccharide nanoparticles described herein may be made of polysaccharides such as dextran, amylose, amylopectin, glycogen, cellulose, arabonixylan, and/or pectin. In certain embodiments, the polysaccharide is dextran. Dextran is a complex, branched glucan (a polysaccharide made of many glucose molecules) composed of chains of varying lengths (from 3-2000 kilodaltons). The straight-chain comprises alpha-1,6-glycosidic linkages between glucose molecules, while branching begins at alpha-1,3 linkages. In some embodiments, dextran nanoparticles are comprised of carboxymethyl dextran.

The polysaccharides that make up the nanoparticles can have a range of molecular weights such as 1 kDa to 1 million kDa (e.g., 1-10 kDa, 10-100 kDa, 100-1000 kDa, or 1000-1,000,000 kDa). The polysaccharide nanoparticles can have an average diameter in a range of 1 nm- 500 nm (e.g., 1-10 nm, 10-25 nm, 25-50 nm, 50-100 nm, or 100-500 nm). The polysaccharide nanoparticles may be relatively monodisperse (e.g., diameters of particles all within a range of 10 nm or less of each other) or more polydisperse.

CSF-1R is a cell surface protein that is a receptor for the colony stimulating factor 1 cytokine. CSF-1R is overexpressed on TAMs. Exemplary CSFR inhibitors comprise pexidartinib, ilorasertib, masitinib, linifanib, ataxilimab, emactuzumab, cabiralizumab, or a combination thereof.

Pexidartinib, sold under the brand name Turalio®, is a kinase inhibitor drug for the treatment of adults with symptomatic tenosynovial giant cell tumor (TGCT) associated with severe morbidity or functional limitations and not amenable to improvement with surgery. Ilorasertib is an inhibitor of the Aurora A, Aurora B and Aurora C kinases and that also inhibits CSF-1R. Masitinib is a kinase inhibitor that also targets CSF-1R on mast cells. Linifanib is also a tyrosine kinase and CSF-1R inhibitor. Ataxilimab, emactuzumab, and cabiralizumab are monoclonal antibodies targeting CSF-1R.

In addition to the CDR-1R inhibitor, the biocompatible hydrogel also encapsulates an effective amount of a platelet-conjugated anti-ICI antibody, wherein the platelet-conjugated anti-ICI antibody activates T-cells.

Immune checkpoints refer to a plurality of inhibitory pathways hardwired into the immune system that are crucial for maintaining self-tolerance and modulating the duration and amplitude of physiological immune responses in peripheral tissues in order to minimize collateral tissue damage. Tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors. In an embodiment, the anti-ICI antibody specifically binds CD25, PD-1, PD-L1, PD-L2, CTLA-4, immunoglobulin receptor (KIR), LAG-3, TIM-3, 4-1BB, 4-1BBL, GITR, CD40, CD40L, OX40, OX40L, CXCR2, B7-H3, B7-H4, BTLA, HVEM, CD28, A2aR, CD27, CD70, TCR ICOS, CD80, CD86, ICOS-L, CD70, Gal-9, VISTA, CD-137,CD155, CD266, PVR, PVR-2, CD47, CD160, NT5E, CD96, TNFRSF18, or a combination comprising one or more of the foregoing. In an embodiment, the anti-ICI antibody is a whole antibody, an antibody fragment, or a peptide.

Exemplary immune checkpoint inhibitors include cemiplimab-rwlc, nivolumab, pembrolizumab, pidilizumab, MEDI-0680, PDR001, REGN2810, and BGB-108, AMP-224, an immunoadhesin, BMS-936559, atezolizumab, YW243.55.S70, MDX-1105, MEDI4736, durvalumab, avelumab, ipilimumab, tremelimumab, BMS-986016, urelumab, TRX518, dacetuzumab, lucatumumab, SEA-CD40, CP-870,893, MED16469, MOXR0916, MSB001078C, or a combination comprising one or more of the foregoing.

In an embodiment, the ICI is a PD-1 binding molecule (e.g., antagonist), and in particular, is an anti-PD-1 antibody (e.g., a human antibody, a humanized antibody, or a chimeric antibody). Exemplary anti-PD-1 antibodies include REGN2810 (cemiplimab), MDX-1106 (nivolumab), MK-3475 (pembrolizumab), CT-011 (pidilizumab), MEDI-0680 (AMP-514), PDR001, and BGB-108 (Tislelizumab). In an embodiment, the PD-1 binding molecule is an immunoadhesin (e.g., an immunoadhesin comprising an extracellular or PD-1 binding portion of PD-L1 or PD-L2 fused to an antibody constant region (e.g., an Fc region of an immunoglobulin sequence). In an embodiment, the PD-1 binding molecule is AMP-224. AMP-224, also known as B7-DCIg, is a PD-L2-Fc fusion soluble receptor described in WO2010/027827 and WO2011/066342.

MDX-1106, also known as MDX-1106-04, ONO-4538, BMS-936558, or nivolumab, is an anti-PD-1 antibody described in WO2006/121168. MK-3475, also known as lambrolizumab (pembrolizumab), is an anti-PD-1 antibody described in WO2009/114335. CT-011, also known as hBAT, hBAT-1, or pidilizumab, is an anti-PD-1 antibody described in WO2009/101611.

In an embodiment, the PD-1 binding molecule is a PD-L1 binding antagonist, and in particular, is an anti-PD-L1 antibody. Exemplary anti-PD-L1 antibodies include MPDL3280A (atezolizumab), YW243.55.S70, MDX-1105, MEDI4736 (durvalumab), and MSB0010718C (avelumab). Antibody YW243.55.S70 is an anti-PD-L1 antibody described in WO 2010/077634. MDX-1105, also known as BMS-936559, is an anti-PD-L1 antibody described in WO2007/005874. MEDI4736 is an anti-PD-L1 monoclonal antibody described in WO2011/066389 and US2013/034559.

Additional ICIs include ipilimumab (anti-CTLA-4), tremelimumab (anti-CTLA-4), BMS-986016 (anti-LAG-3), urelumab (anti-4-1BB), MSB001078C (anti-4-1BB), TRX51 (anti-GITR), dacetuzumab (anti-CD40), lucatumumab (anti-CD40), SEA-CD40 (anti-CD40), CP-870,893 (anti-CD40), MED16469 (OX40), and MOXR0916 (OX40).

In a specific aspect, the anti-ICI antibody is an anti-PD-1 antibody.

The anti-ICI antibody is conjugated to platelets. Platelets can be purified from whole blood using centrifugation, for example. In order to conjugate the anti-ICI antibody to the platelets, the anti-ICI antibody can be chemically modified with, for example, a bifunctional linker. In an embodiment, the bifunctional linker includes SMCC (succinimidyl-4-(N-maleimidomethyl)cyclohexane-1-caboxylate), MBS (m-maleimidobenzoyl-N-hydroxysuccinimide ester), sulfo-MBS (m-maleimidobenzoyl-N-hydroxysulfosuccinimide ester), GMBS (N-γ-maleimidobutyryloxysuccinimide ester), sulfo-GMBS (N-γ-Maleimidobutyryloxysulfosuccinimide ester, EMCH (N-(ε-maleimidocaproic acid) hydrazide), EMCS (N-(ε-maleimidocaproyloxy)succinimide ester), sulfo-EMCS N-(ε-maleimidocaproyloxy) sulfo succinimide ester), PMPI (N-(ρ-maleimidophenyl) isocyanate), SIAB (N-succinimidyl(4-iodoacetyl)aminobenzoate), SMPB (succinimidyl 4-(ρ-maleimidophenyl) butyrate), sulfo-SIAB (N-sulfosuccinimidyl(4-iodoacetyl)aminobenzoate), sulfo-SMCC (sulfosuccinimidyl 4-(N-maleimidomethyl) cyclohexane-1-carboxylate), sulfo-SMBP (sulfo succinimidyl 4-(ρ-maleimidophenyl) butyrate), EDC (1-ethyl-3-(3-dimethylaminopropyl) carbodiimide hydrochloride), or MAL-PEGSCM (maleimide PEG succinimidyl carboxymethyl). In an embodiment, the bifunctional linker comprises SMCC or an NHS ester. SMCC is a hetero-bifunctional linker that contains N-hydroxysuccinimide (NHS) ester and maleimide groups that allow covalent conjugation of amine-and sulfhydryl-containing molecules. NHS esters react with primary amines at pH 7-9 to form amide bonds, while maleimides react with sulfhydryl groups at pH 6.5-7.5 to form stable thioether bonds.

Also disclosed herein is a method of treating a post-surgical tumor site in a subject, the method comprising depositing at the post-surgical tumor site an effective amount of the depot formulation described herein. The depot formulation is expected to reduce tumor recurrence, leading to increased survival rates. An effective amount or “therapeutically effective amount” of the depot formulation means an amount effective when administered to a subject, which provides a therapeutic benefit. The therapeutic benefit can include amelioration of symptoms, a decrease in disease progression, or inhibiting the development of the disease. In particular, the therapeutic benefit includes prevention, treatment, and/or inhibition of post-surgery tumor recurrence in a subject.

Exemplary solid tumors include bladder, breast, cervix, colon, rectal, endometrial, kidney, oral, liver, lung, melanoma, non-small cell lung cancer, ovarian, pancreatic, prostate, sarcoma, small cell lung cancer, and thyroid, for example.

The method can further comprise systemically administering to the subject a systemic formulation comprising platelet-conjugated anti-ICI antibodies, wherein the anti-ICI antibodies in the depot formulation and the systemic formulation are the same or different. The systemic formulation comprises an effective amount of the platelet-conjugated anti-ICI antibodies.

In an aspect, a method of treating a post-surgical tumor site in a subject comprises depositing at the post-surgical tumor site an effective amount of a depot pharmaceutical formulation, comprising a biocompatible hydrogel, the hydrogel encapsulating an effective amount of a colony-stimulating factor receptor (CSFR) inhibitor, wherein the CSFR inhibitor blocks CSF receptors on tumor associated macrophages (TAMs), and systemically administering a systemic formulation comprising a platelet-conjugated anti-immune checkpoint inhibitor (ICI) antibody, wherein the platelet-conjugated anti-ICI antibody activates T-cells.

The invention is further illustrated by the following non-limiting examples.

### EXAMPLES

### Materials and Methods

Antibodies and cells: The mouse melanoma B16F10 cells and mouse CT26 cells were tagged with luciferase for in vivo bioluminescence imaging. The B16F10, NIH/3T3 and Raw 264.7 cells were purchased from ATCC. Luciferase-expressed B16F 10 and CT26 cells were obtained from Imanis Life Sciences Inc. Cells were cultured in the CO2 incubator (Fisher) at 37° C. with 5% CO₂ and 90% relative humidity. The cells were sub-cultured about every 2 days at 80% confluence. The antibodies used in this study were summarized as follows (company, clone, category number): GoInVivo™ Purified anti-mouse CD279 (PD-1) (BioLegend®, RMP1-14, 114114), Fluorescein isothiocyanate (FITC)-anti-mouse CD45 (BioLegend®, 30-F11, 103108), APC-anti-mouse F4/80 (BioLegend®, BM8, 123116), PerCP/Cy5.5-anti-human/mouse CD11b (BioLegend®, M1/70, 101227), APC-anti-mouse CD3 (BioLegend®, 17A2, 100236), FITC-anti-mouse CD4 (BioLegend®, GK1.5, 100406), PE-anti-mouse CD8a (BioLegend, 53-6.7, 100708), FITC-anti-mouse IFNρ (BioLegend®, XMG1.2, 505806), PerCP/Cy5.5-anti-human/mouse Granzyme B (BioLegend®, QA16A02, 372212), PE-anti-mouse CD45 (BioLegend®, 30-F11, 103106), FITC-anti-human/mouse CD11b (BioLegend®, M1/70, 101206), Alexa Fluor® 594 anti-mouse CD8a (BioLegend®, 53-6.7, 100758), Alexa Fluor® 647 anti-mouse F4/80 (BioLegend®, BM8, 123121), PE-anti-mouse CD62P (BioLegend®, RMP-1, 124807), PE-anti-mouse CD41 (BioLegend®, MWReg30, 133905), FITC-anti-mouse CD9 (BioLegend®, MZ3, 148305), FITC-anti-mouse CD61 (BioLegend®, 2C9.G2, 104305). All antibody dilutions were performed following the manufacture's guidance. FlowJo software was used to analyze flow cytometry data.

Preparation and characterization of PLX-NP: Dextran was modified with pyridinium p-toluenesulfonate (PPTS) and 2-ethoxypropene for the preparation of nanoparticles. Briefly, 1 g dextran was dissolved in 10 ml anhydrous dimethyl sulfoxide, and 0.062 mmol PPTS (Sigma Aldrich) and 37 mmol 2-ethoxypropene (Matrix Scientific) were added to the dextran solution during stirring. After 30 min, the reaction was quenched by adding 1 ml triethylamine (Sigma Aldrich) during stirring at room temperature, resulting in 2-ethoxypropene-modified dextran (designated m-dextran). m-dextran was then precipitated in the basic water and collected by centrifugation.

To prepare PLX-NP, 10 mg m-dextran and 0.5 mg PLX were firstly dissolved in 2 ml dichloromethane (DCM). Afterwards, 4 ml 3% (w/v) poly (vinyl alcohol) (PVA) solution was then slowly added to the DCM solution followed by sonication for emulsification. Then, the emulsion was added to 20 ml 0.3% (w/v) PVA solution and stirred for one hour for solvent evaporation. The nanoparticles were collected by centrifugation at 14,500 rpm for 45 min. The resulting PLX-NP was analyzed by dynamic light scattering (DLS) to determine the average size, and the morphology of nanoparticles was characterized by TEM. To study the in vitro release property of PLX, PLX-NP was suspended in 3 ml phosphate-buffered saline (PBS, pH 6.5) with 0.1% Tween 80 and loaded into a 3 ml 20,000 MWCO dialysis cassette (Thermo scientific). The cassette was placed into a container with 4 L PBS with 0.1% v/v Tween 80, and at predetermined time points, 10 μl supernatant was collected, dissolved by acetonitrile, and the concentration was analyzed by high-performance liquid chromatography (HPLC). To further test the cytotoxicity of PLX-NP against macrophages and normal cells, the cell viability of Raw 264.7 and NIH/3T3 cells treated with different concentrations (0 to 125 μg/ml) of PLX-NP for 24 h was determined by MTT assay.

Preparation and characterization of anti-PD-1-conjugated platelets: The mouse platelets were purified from whole mouse blood collected by retro-orbital bleeding. Afterwards, the blood was centrifuged for 20 min at 100 g, followed by centrifugation for 20 min at 800 g. The platelets were collected and suspended in PBS with the addition of 1 μM PGE1 to prevent platelet activation. To conjugate aPD-1 antibodies on the surface of platelets, aPD-1 antibodies were first reacted with SMCC linkers for 2 h at 4° C. at a molar ratio of 1:1.2. And then, the excess SMCC linkers were discarded by centrifugation at 14,000 rpm for 10 min at 4° C., using 3000 KDa MWCO ultrafiltration tubes. The synthesized SMCC-aPD-1 was added into platelets and stirred at room temperature for 1 h to obtain P-aPD-1. The excess antibodies were removed by centrifugation at 1,500 g for 20 min. To characterize the conjugation efficacy of aPD- 1 with platelets, P-aPD-1 was subjected to 0.1% Triton™-X100 buffer to release aPD-1, and the amount of aPD-1 was determined by ELISA kit (Rat IgG total ELISA Kit, Invitrogen).

To demonstrate the successful conjugation of aPD-1 on the surface of platelets, confocal microscopy (Nikon A1RS) and flow cytometry (ThermoFisher Attune™) were performed. Briefly, aPD-1 was stained by FITC, and platelets were stained by Wheat Germ Agglutinin 594 (WGA 594). And then, the functionality of aPD-1 conjugated platelets was studied by two assays: collagen binding assay and surface antigen expression study. First, collagen from the human placenta (Sigma) is reconstituted to a concentration of 1.0 mg/ml and was added to a confocal dish for incubation overnight at 4° C. The coated confocal dishes and uncoated confocal dishes were further blocked with 1 ml 2% (w/v) bovine serum albumin in PBS for two hours and washed with PBS. Rhodamine B-labeled naïve platelets and P-aPD-1 were then added to the dishes and incubated for 5 min. The unbinding platelets and P-aPD-1 were washed with PBS, and then the dishes were visualized under the confocal microscope. The surface protein expression of P-aPD-1 was also investigated by flow cytometry by staining with various antibodies (CD61, CD41, CD9), compared with unmodified platelets. Furthermore, the platelet activation marker CD62P was characterized by flow cytometry after P-aPD-1 was treated with thrombin.

Preparation of alginate-based hydrogel: The alginate-based hydrogel was formed by adding 10 μl 100 mg/ml CaCl₂ solution into 200 μl 10 mg/ml alginate solution in HEPES buffered saline. To study the in vivo degradation rate of alginate-based hydrogel, alginate was conjugated with Cy5. Briefly, 50 mg sodium alginate was dissolved in 5 ml of HEPES buffer (50 mM, pH=5), 14 mg NHS, 116 mg EDC, and 18 mg NH2-PEG3-N3, and the mixture were stirred for 30 min at room temperature. Then the pH of the solution was adjusted to 7.5-8.0 and reacted overnight at room temperature. The synthesized alginate-N3 was purified by 3-day dialysis against water. The 100 μl of 1% (w/v) solution of alginate-N3 was incubated with 30 μl of 1 mM Cy5-DBCO for four hours at 37° C. And the final product was purified using dialysis against water. The synthesized alginate-Cy5 was mixed with unreacted alginate at a volume ratio of 1:1 to form Cy5-labeled hydrogel. The Cy5-labeled hydrogel was implanted into the C57BL/6 mice subcutaneously, and the fluorescence signals were monitored by IVIS (Perkin Elmer).

Characterization of hydrogel loading with PLX-NP and P-aPD-1: The predetermined amounts of PLX-NP and P-aPD-1 were suspended into 50 μl HEPES buffered saline and mixed with 10 μl 100 mg/ml CaCl2 solution, adding to 200 μl 10 mg/ml alginate solution in HEPES buffered saline to form PLX-NP-P-aPD-1@Gel. The scanning electron microscope (SEM) was first performed to visualize the morphology of PLX-NP-P-aPD-1@gel, and confocal microscopy was also used to characterize the successful loading of NP and P-aPD-1 in the hydrogel. WGA 594 labeled P-aPD-1 and FITC-loaded NP were synthesized and loaded into the alginate hydrogel, and the hydrogel was then observed under the confocal microscope.

To examine the loading capacity of alginate-based hydrogel, different volumes of suspension containing a fixed amount of PLX-NP and P-aPD-1 were loaded into a 200 μl alginate solution in a 96-well plate. After 3 min, hydrogels were removed from wells, and the P-aPD-1 was counted using hemocytometers under the microscope, and the remaining amounts of PLX were determined by HPLC.

To study the platelets and aPD-1 release from hydrogel, a hydrogel containing 1×10⁸ P-aPD-1 was placed into a 40 μm cell strainer in a 6-well plate and submerged by 5 ml PBS. Afterwards, 1 U/ml thrombin was added to trigger the activation of platelets. At predetermined time points, 50 μl samples were collected, and the same amount of PBS was added back to the wells. The platelets in collected samples were counted using hemocytometers under the microscope. Then, the collected samples were centrifuged for 20 min at 800 g, and the concentration of aPD-1 in the supernatant was detected by rat total IgG ELISA kit. The P-aPD-1 without activation was used as a control group.

In vivo macrophage depletion ability of PLX-NP@Gel: The C57BL/6 mice (Male, aged 5-7 weeks) were purchased from Jackson laboratory. The animal study protocol was approved by the Institutional Animal Care and Use Committee at the University of Wisconsin-Madison. To build the in vivo mouse B16F10 melanoma recurrence model, 2.5×10⁵ B16F10 cells were injected into the right flank of C57BL/6 mice subcutaneously. Once the tumor size reached about 150 mm³, about 95% of tumors were resected (leaving about 5% tumors). Different treatments were then applied to the tumor cavities, including saline, NP@Gel, free PLX, PLX-NP, and PLX-NP@Gel. One week after the treatments, tumors were collected for the following studies. Collected tumor tissues were digested by collagenase and then were dissociated by tissue dissociator (gentalMACS) to obtain single-cell suspension. The cell suspension was stained with FITC-anti-mouse CD45, APC-anti-mouse F4/80, PerCP/Cy5.5-anti-human/mouse CD11b, FITC-anti-mouse CD4, PE-anti-mouse CD8a, FITC-anti-mouse IFNγ antibodies and analyzed using flow cytometry. Furthermore, the collected tumors were embedded in optimal cutting temperature (OCT) compound and frozen in a −80° C. freezer for sections. To directly visualize the macrophages and T cells, the section slides were stained by the Alexa Fluor® 594 anti-mouse CD8a, Alexa Fluor® 647 anti-mouse F4/80 antibodies, and Hoechst 33342 trihydrochloride (Invitrogen, H3570), and then were imaged by the confocal microscope.

In vivo anti-tumor efficacy of PLX-NP-P-aPD-1@Gel: The in vivo mouse B16F10 melanoma recurrence model was established as described before. The in vivo mouse CT26 colon cancer recurrence model was established by subcutaneously injecting one million CT26 cells in the right flank of the mouse. Both B16F10 and CT26 cells were tagged with luciferase for in vivo bioluminescence imaging. For both tumor models, different treatments were applied to the tumor cavities, including saline, NP-P@Gel (blank nanoparticle and unmodified platelets co-loaded hydrogel), PLX-aPD-1@Gel (free PLX and aPD-1 co-loaded hydrogel), PLX-NP@Gel (PLX-NP loaded hydrogel), P-aPD-1@Gel (P-aPD-1 loaded hydrogel), PLX-NP+P-aPD-1 (free PLX-NP and P-aPD-1), PLX-NP-P-aPD-1@Gel (PLX-NP and P-aPD-1 co-loaded hydrogel). The dose of aPD-1 was 0.1 mg/kg per mouse, and the dose of PLX was 5 mg/kg per mouse. From day 0, at predetermined days, the bioluminescence signals of the resected tumor tissues were monitored by IVIS, after intraperitoneally injecting 150 mg/kg D-luciferin per mice in 100 μl PBS. Mice were imaged after 5 min with 0.5-second exposure. Bioluminescence images were analyzed using Living Image Software v.4.3.1 (Perkin Elmer). The weight and survival of mice were monitored during the time-course of treatment. Once the tumor volume was larger than 1.5 cm³ (calculated based on the equation: length×width²×0.5), mice were euthanized following the animal protocols.

To investigate whether P-aPD-1 could be activated at the tumor site, WGA 594-labeled P-aPD-1 was loaded into the hydrogel. One week after subcutaneous B16F10 tumor implantation, the tumor was surgically resected, and hydrogel was put into the tumor cavity beside the residue tumor. After three days, the residue tumor was collected for the frozen section. The section slides were imaged under the confocal microscope.

To study in vivo macrophage depletion ability and enhanced T cell infiltration of NP-PLX-P-aPD-1@Gel, the resection tumor model was established as above-mentioned and treated with saline, NP-P@Gel, PLX-aPD-1@Gel, PLX-NP@Gel, P-aPD-1@Gel, PLX-NP+P-aPD-1, PLX-NP-P-aPD-1@Gel. After one week, tumors were collected and digested by collagenase and then were dissociated by a tissue dissociator to obtain single-cell suspension. The cell suspension was stained with PE-anti-mouse CD45, FITC-anti-human/mouse CD11b, APC-anti-mouse F4/80, APC-anti-mouse CD3, FITC-anti-mouse CD4, PE-anti-mouse CD8a, and PerCP/Cy5.5-anti-human/mouse Granzyme B antibodies, and analyzed using flow cytometry. Furthermore, the collected tumors were also embedded in OCT for frozen section. The section slides were stained by the Alexa Fluor® 594 anti-mouse CD8a, Alexa Fluor® 647 anti-mouse F4/80 antibodies, and Hoechst 33342trihydrochloride for observation under the confocal microscope. Moreover, to study the cytokine generation after treatments, tumor tissues were collected after one-week treatments. The tumor tissues were resuspended in NP40 Cell Lysis Buffer (Alfa Aesar) at 4° C. and then were mechanically ground. The homogenate was centrifuged for 10 min at 6,000 rpm, at 4° C. to collect the supernatant. Afterwards, 10 μl supernatant was used to be detected using corresponding cytokine ELISA kits following the manufacture's guidance.

In vivo anti-tumor efficacy of PLX-NP@Gel and systemic injection of P-aPD-1: To build the in vivo mouse B16F10 melanoma recurrence model, 2.5×10⁵ B16F10 cells were injected into the right flank of C57BL/6 mice subcutaneously. Once the tumor size reached about 150 mm³, surgery was performed and different treatments were applied to the tumor cavities, including saline, PLX-NP@Gel, PLX-NP@Gel with intravenous injection of free aPD-1 antibodies every other day for three times starting from day 0, and PLX-NP@Gel with intravenous injection of P-aPD-1 every other day for three times starting from day 0. The dose for aPD-1 was 0.5 mg/kg per mouse, and the dose for PLX was 5 mg/kg per mouse. From day 0 at predetermined days, the volumes of the tumor tissues were measured using a digital caliper and calculated based on the equation: length×width²×0.5. The survival of mice was recorded for 50 days. Once the tumor volume was larger than 1.5mm³ , mice were euthanized following the animal protocols. Moreover, the tumor tissues were collected for weight measure at three weeks after the hydrogel implantation, and the representative tumor tissues from each group were imaged. To study in vivo macrophage depletion ability and enhanced T cell infiltration of different groups, the flow cytometry and the ELISA assays for IFNγ and TNFα were performed as mentioned above.

Statistics: All the results are shown as mean ±s.d. The GraphPad Prism software was used to perform statistical analysis. One-way analysis of variance (ANOVA) was used to compare multiple groups (>two groups) statistically. Log-rank (Mantel-Cox) test was performed for the statistical analysis for the survival study. A P value lower than 0.05 (*P<0.05) was considered as the threshold for statistical significance among control groups and experimental groups; *P<0.05, **P<0.01, ***P<0.001.

### EXAMPLE 1: PREPARATION AND CHARACTERIZATION OF PLX-NP-P-APD-1@GEL

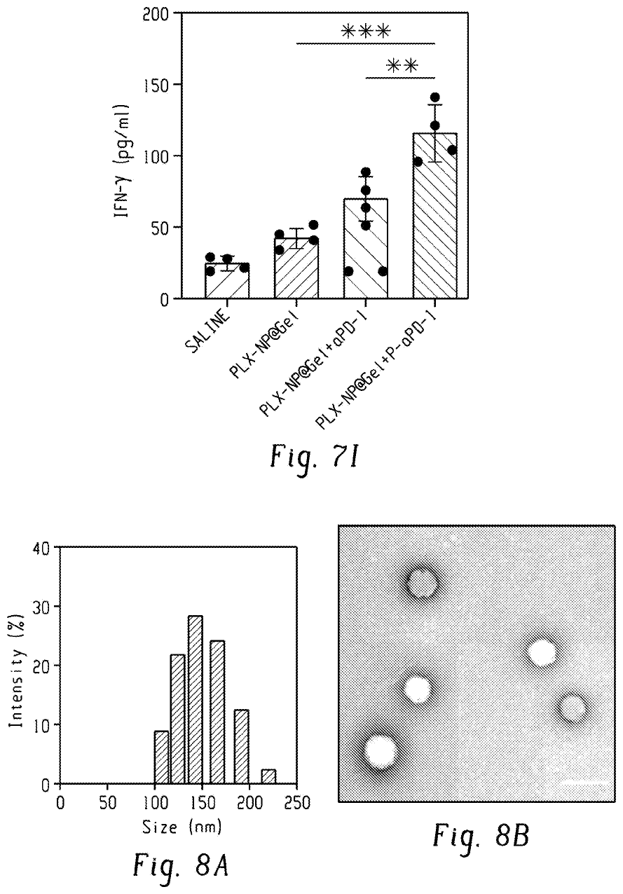

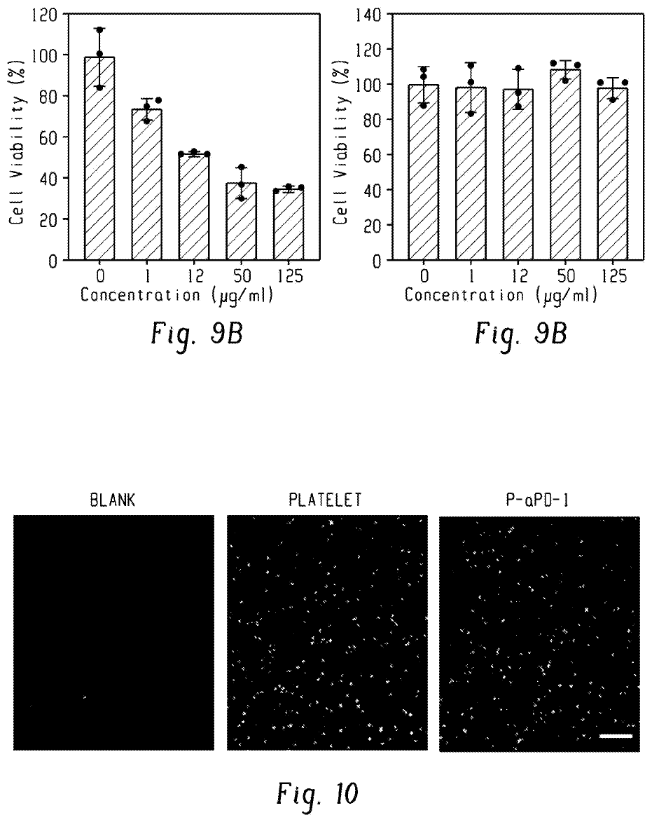

PLX-loaded dextran nanoparticles were prepared by the single-emulsion method. The average size of the PLX-NP was determined to be 145 nm by dynamic light scattering (DLS) (FIG. 8A). Additionally, representative transmission electron microscopy (TEM) image of the PLX-NP showed the monodispersed PLX-NP with spherical morphologies (FIG. 8B). The in vitro release of PLX from PLX-NP was then investigated at pH 6.5 to mimic the pH in the acidic tumor microenvironment. As shown in FIG. 1B, PLX-NP displayed a sustained release manner of PLX, and the cumulative release percentage reached 50.3% by day 5. Moreover, to study the in vitro macrophage cytotoxicity of PLX-NP, a cellular MTT assay was performed on RAW264.7 cells showing that PLX-NP displayed dose-dependent macrophage-specific cytotoxicity, while with no significant impact on the viability of NIH/3T3 fibroblasts (FIG. 9A-B).

Anti-PD-1 antibody-conjugated platelets were prepared by covalently coupling the amine groups on aPD-1 antibodies with the thiol groups on the surface of platelets using sulfo-SMCC linkers. The successful conjugation was determined using confocal microscopy, as evidenced by the overlap between WGA 594-labeled platelets and fluorescein isothiocyanate (FITC)-labeled aPD-1 antibodies (FIG. 1C). The conjugated amount of aPD-1 was set as 0.1 pg/platelet according to previous studies, which showed negligible cytotoxicity against platelets. To further affirm that the aPD-1 conjugation would not affect the bio-functionality of platelets, a collagen binding assay was performed. As shown in FIG. 10, the collagen binding ability of P-aPD-1 did not change after aPD-1 decoration, compared with naïve platelets. Furthermore, the expression of platelet surface markers, including CD61, CD41, CD9, and CD62P, did not change significantly compared with naïve platelets (FIG. 11), indicating P-aPD-1 reserved intrinsic properties of platelets.

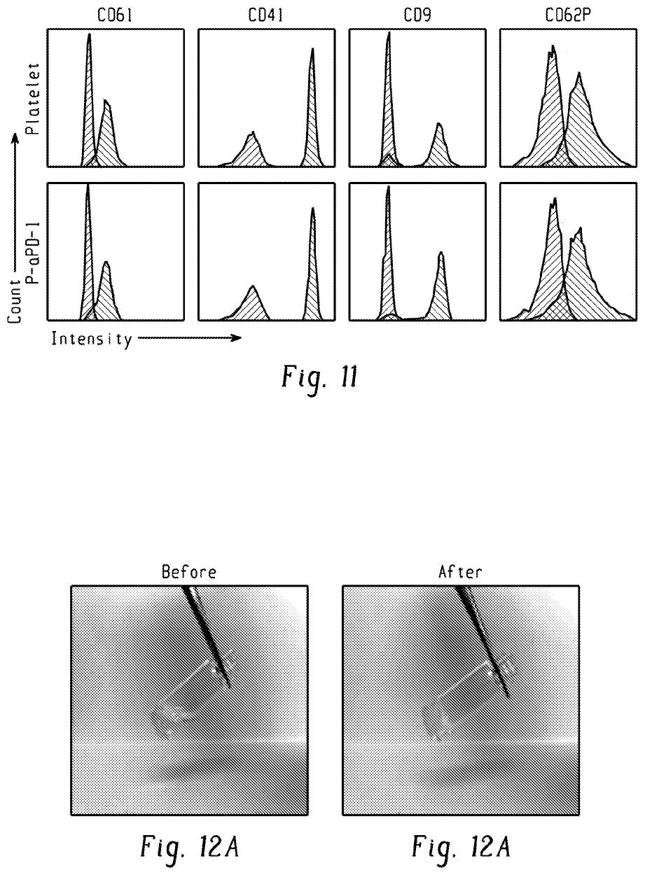

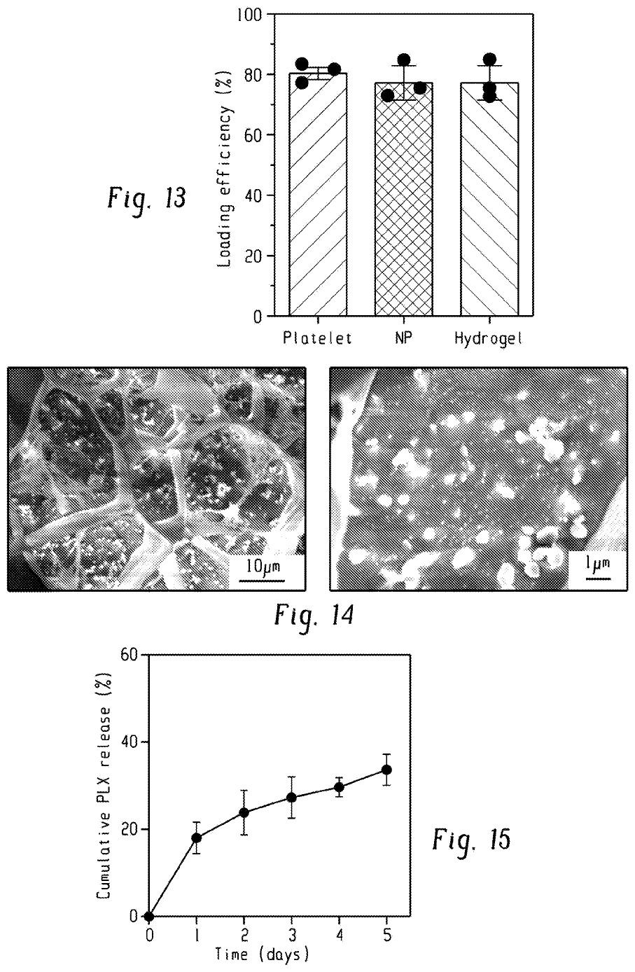

Then, PLX-NP and P-aPD-1 were loaded into a biocompatible alginate hydrogel. As shown in FIG. 12, the alginate solution underwent a quick sol-to-gel transition after the addition of Ca²⁺ solutions. To investigate the loading efficiency of PLX-NP and P-aPD-1 in the hydrogel, various amounts of Ca²+ solution with PLX-NP and P-aPD-1 were applied to form alginate hydrogel. 80.9% and 77.8% loading efficiency of P-aPD-1 and PLX-NP were achieved, respectively, when the ratio of Ca²+ solution containing P-aPD-1 and PLX-NP and alginate solution was set as 1:3.3 (FIGS. 13A, 13B). The co-existence of PLX-NP and P-aPD-1 in the hydrogel was visualized by confocal imaging and cryo-scanning electron microscope (SEM), where both PLX-NP and P-aPD-1 were embedded in a networking structural hydrogel (FIGS. 1D, 1E; FIG. 14).

Next, the release profiles of both platelets and aPD-1 antibodies from the hydrogel were characterized in transwell devices. As shown in FIG. 1F-G, sustained-release was observed for both platelets and aPD-1 antibodies, in which 84% of platelets and 60% of aPD-1 antibodies were released within 24 hours. Also, the PLX was gradually released from the hydrogel-based delivery system in vivo, and approximately 33.7% drug was released after 5 days (FIG. 15). To evaluate the in vivo degradation of the hydrogel, the C57BL/6 mice were surgically implanted with Cy5-labeled alginate hydrogel. The hydrogel displayed superior biocompatibility and biodegradability in vivo, as evidenced by a decayed fluorescent signal during the time-course of implantation (FIGS. 1H, 1I).

### EXAMPLE 2: IN VIVO TUMOR ASSOCIATED MACROPHAGES DEPLETION CAPABILITY OF PLX-NP-LOADED HYDROGEL

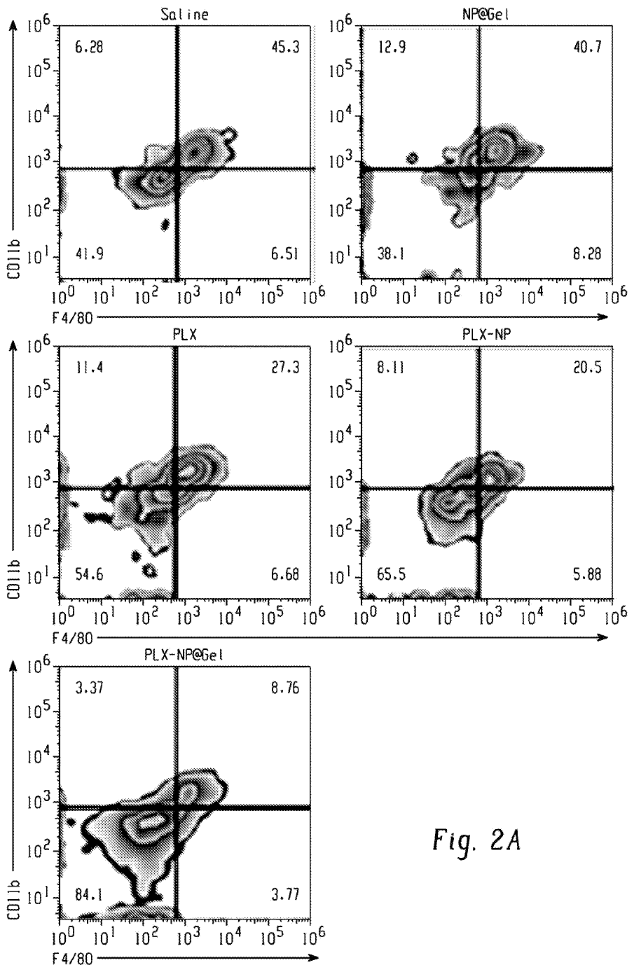

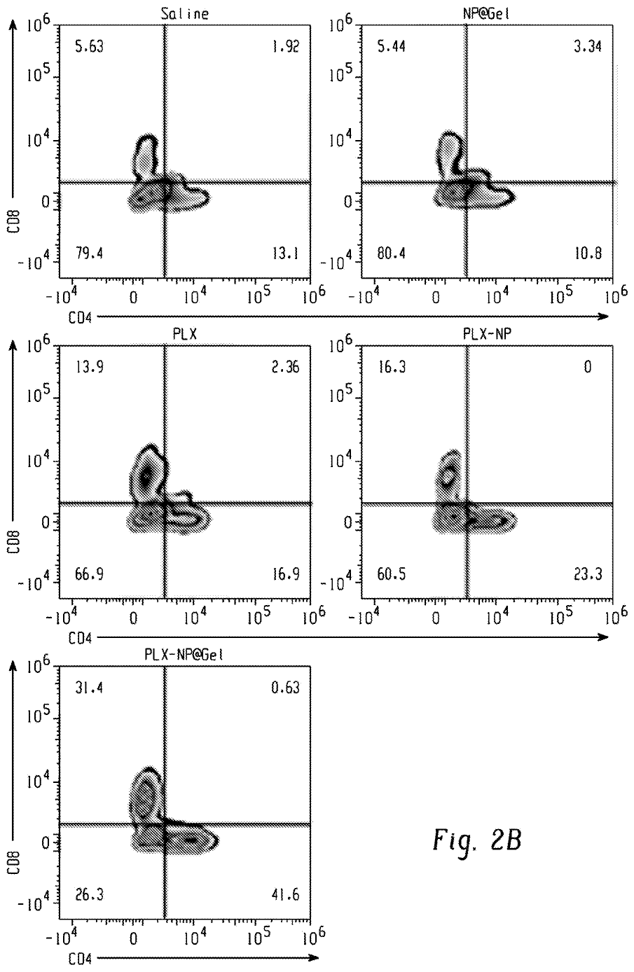

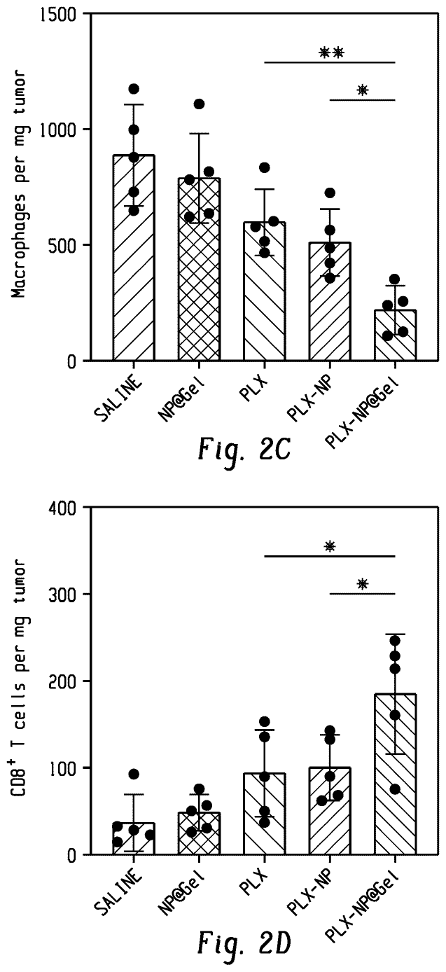

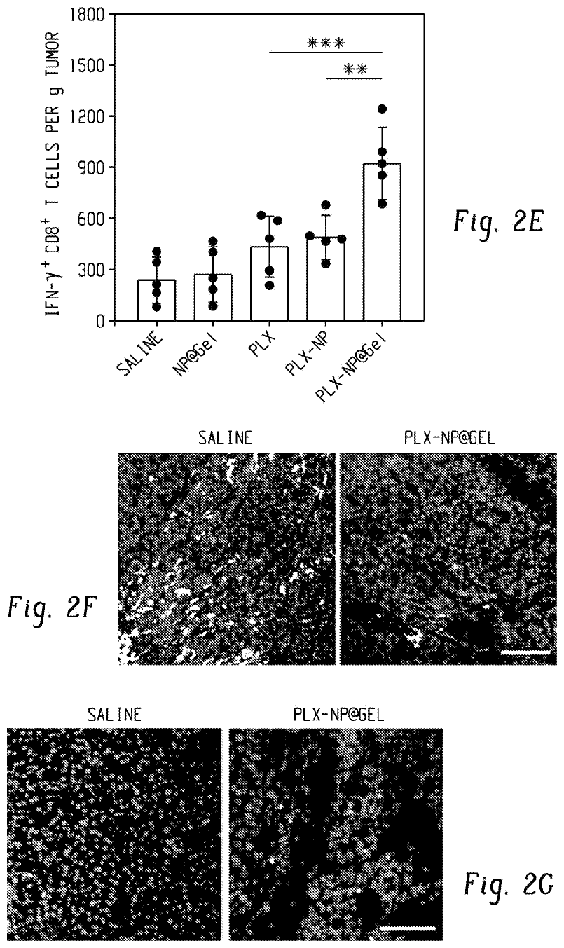

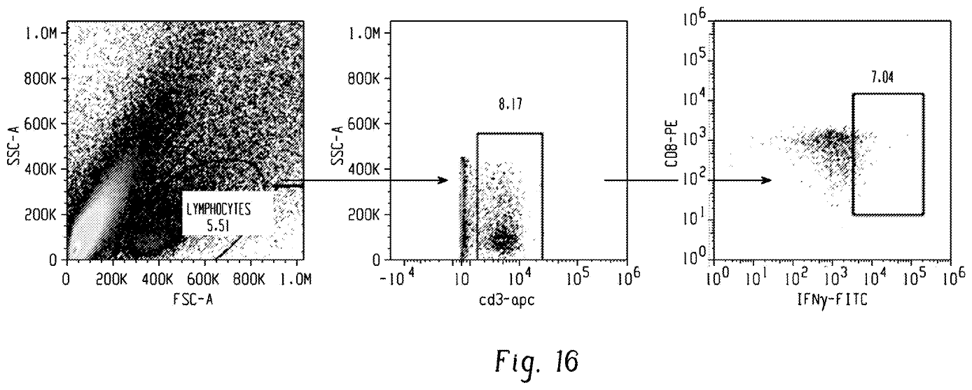

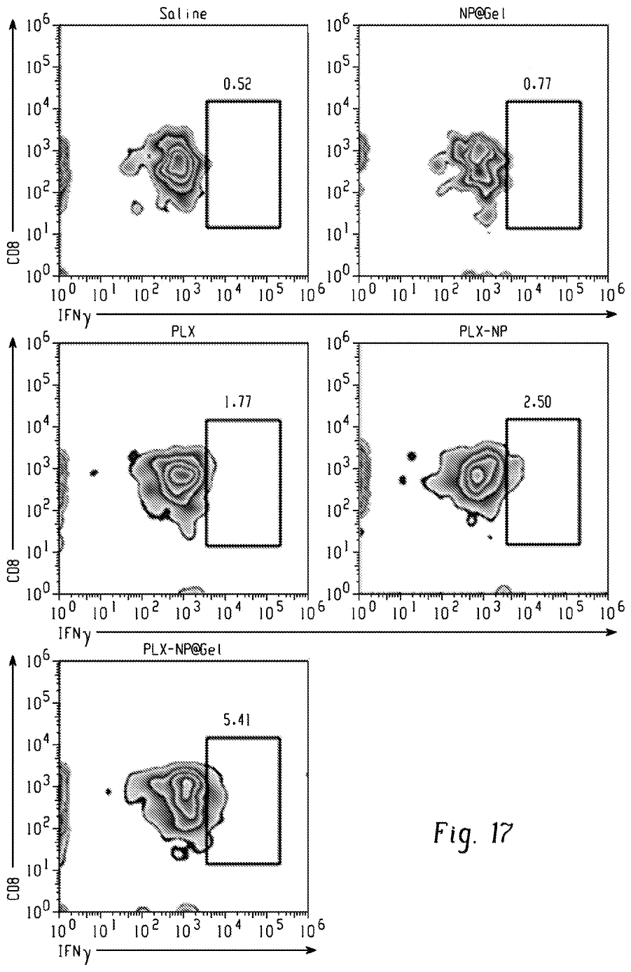

Whether PLX-NP@Gel implanted at the tumor surgery cavity has the ability to deplete TAMs and enhance CD8⁺ T cell infiltration was investigated. The melanoma recurrence mouse model was built by surgery, once the tumor size reached about 150 mm³. Afterwards, the mice were treated with saline, blank nanoparticle-loaded hydrogel (NP@Gel), free PLX, PLX-NP, PLX-NP-loaded hydrogel (PLX-NP@Gel) and were collected for flow cytometry analysis. As shown in FIG. 2A, the percentages of macrophages in the tumor tissues treated with all PLX formulations were decreased compared with saline and NP@Gel groups, demonstrating the TAMs depletion capability of PLX by blocking CSF1 receptors. There was no substantial difference in TAMs between PLX and PLX-NP groups, which could be attributed to the quick clearance of both free PLX and PLX-NP. Furthermore, PLX-NP@Gel displayed a significantly enhanced potency compared to free PLX and PLX-NP groups, as evidenced by 2.3-fold and 1.8-fold decreases in the density of TAMs, highlighting the superiority of hydrogel as a local reservoir for controlled release of PLX-NP for enhanced efficacy (FIG. 2C). In addition, the increased infiltration of CD8⁺ T cells was observed as a result of enhanced TAMs depletion. As shown in FIGS. 2B and 2D, PLX-NP@Gel treatment induced 1.84-fold and 1.96-fold increases in CD8⁺ T cells density compared to PLX-NP and PLX groups. Moreover, the density of IFNγ⁺ CD8⁺ T cells in PLX-NP@Gel group was 1.8-fold and 2.1-fold higher than that in PLX-NP and PLX groups (FIGS. 2E, 16, and 17).

To directly visualize the TAMs depletion and CD8⁺ T cells infiltration, the tumor tissues were collected and sectioned for immunostaining. A substantial decrease of TAMs and increase of infiltrated CD8⁺ T cells were observed in confocal images in comparison of saline and PLX-NP@Gel groups (FIGS. 2F, 2G), which were consistent with the flow cytometry quantitative analysis of TAMs and CD8⁺ T cells, respectively (FIGS. 2C, 2D). Collectively, the PLX-NP@Gel displayed a potent TAMs depletion capability for modulating immunosuppressive tumor microenvironment, leading to the enhanced infiltration of CD8⁺ T cells for potentially improved tumor immunotherapy.

### EXAMPLE 3: IN VIVO ANTI-TUMOR EFFICACY OF PLX-NP-P-APD-1@GEL IN TUMOR RECURRENCE MODEL OF COLON CANCER AND MELANOMA

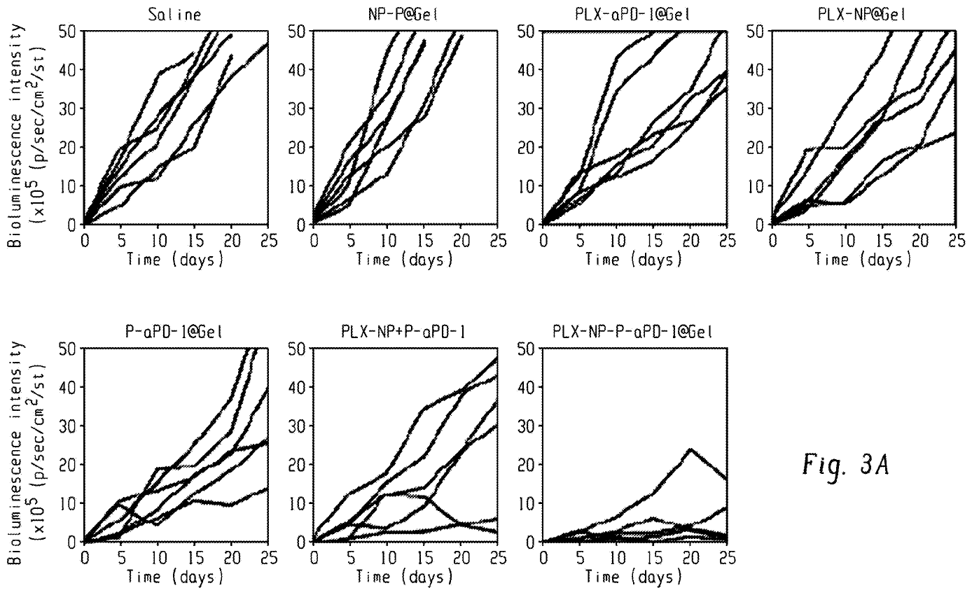

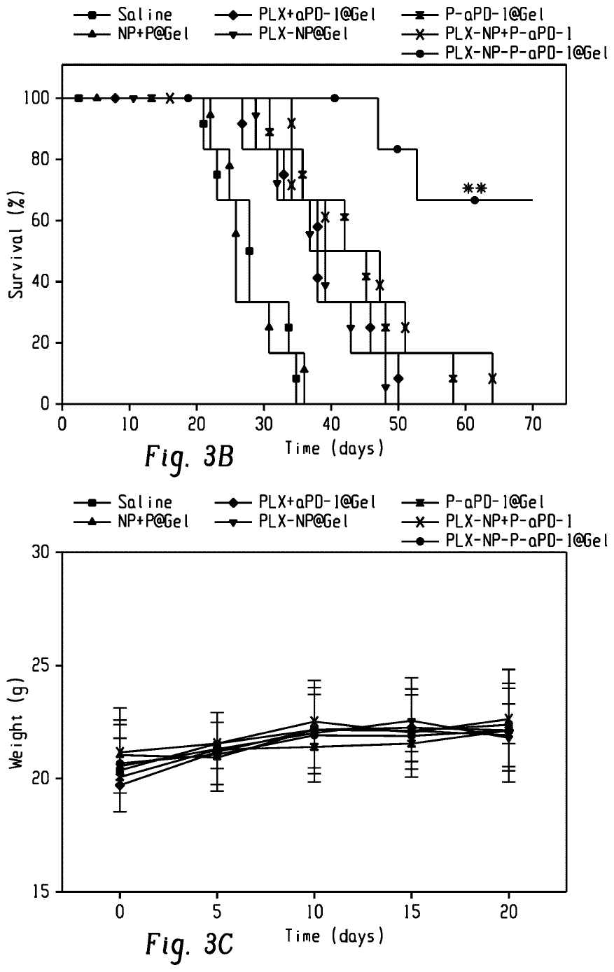

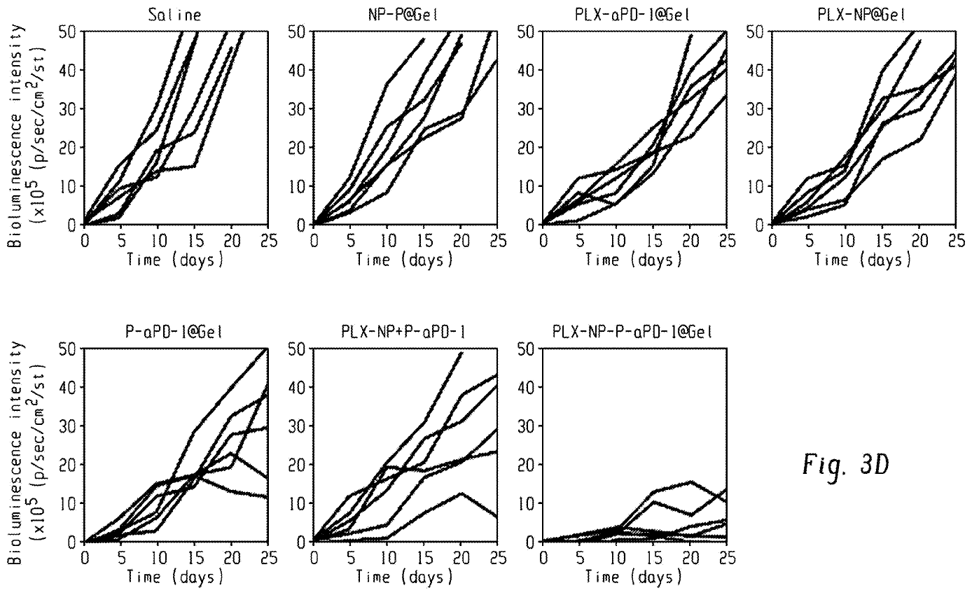

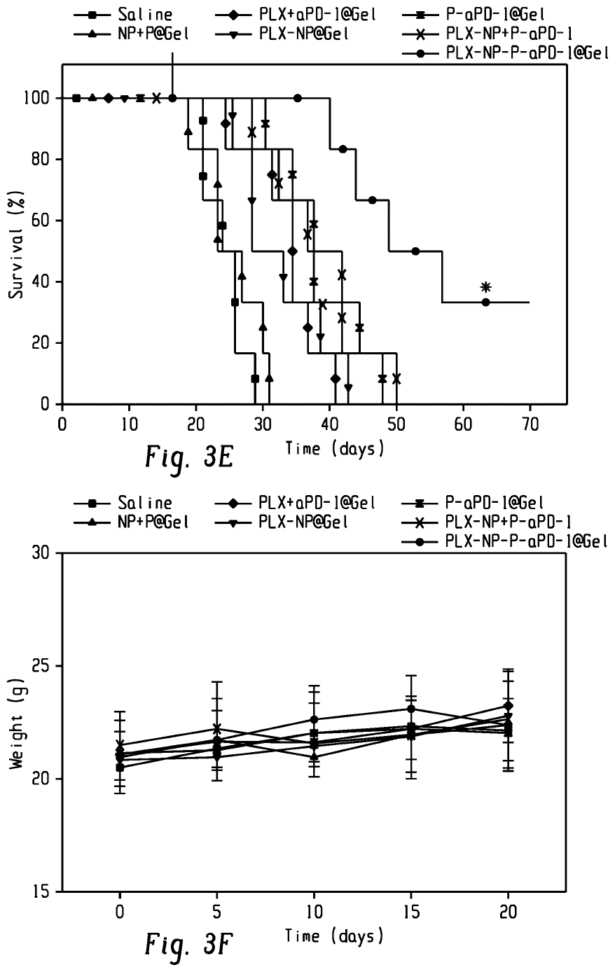

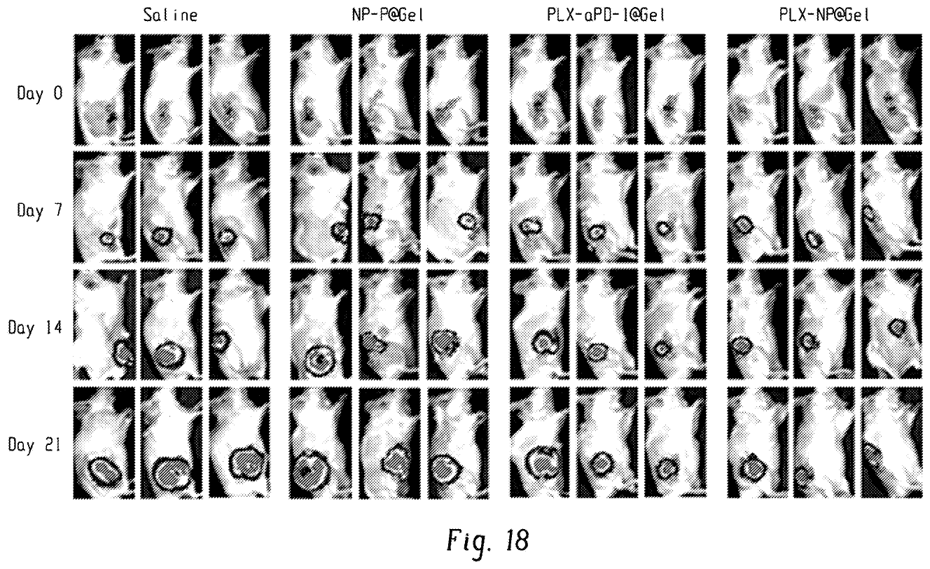

To further demonstrate the immunotherapeutic efficacy of PLX-NP-P-aPD-1@Gel, first, the CT26 colon cancer recurrence mouse model was established and treated with various treatment groups. The recurrent tumor growth was monitored by measuring bioluminescence signals from luciferase-tagged CT26 cells during the time-course of treatment, which displayed a negligible difference of the resected tumors initially (FIG. 18). Afterwards, various treatments were applied to the postoperative mice, including saline, NP-P@Gel (blank nanoparticle and unmodified platelets co-loaded hydrogel), PLX-aPD-1@Gel (free PLX and aPD-1 co-loaded hydrogel), PLX-NP@Gel (PLX-NP loaded hydrogel), P-aPD-1@Gel (P-aPD-1 loaded hydrogel), PLX-NP+P-aPD-1 (free PLX-NP and P-aPD-1), PLX-NP-P-aPD-1@Gel (PLX-NP and P-aPD-1 co-loaded hydrogel) at the doses of 5 mg/kg PLX and 0.1 mg/kg aPD-1. Bioluminescence signals of tumors were utilized to monitor the tumor growth during the time-course of treatment. The tumor growth of mice in the saline group and NP-P@Gel group was barely inhibited, where all the mice died within 36 days (FIG. 3A). Moreover, compared with moderate anti-tumor effects of the PLX-aPD-1@Gel, PLX-NP@Gel, P-aPD-1@Gel, and PLX-NP+P-aPD-1, the tumor growth was remarkably suppressed in the PLX-NP-P-aPD-1@Gel-treated mice, demonstrating the potent therapeutic efficiency of PLX-NP-P-aPD-1@Gel in colon cancer recurrence model. Moreover, PLX-NP-P-aPD-1@Gel significantly prolonged the survival of the mice, with over 60% of the mice alive within 70 days (FIG. 3B). Furthermore, the biocompatibility of PLX-NP-P-aPD-1@Gel with no obvious potential toxicity was proved by no significant weight loss of mice (FIG. 3C).

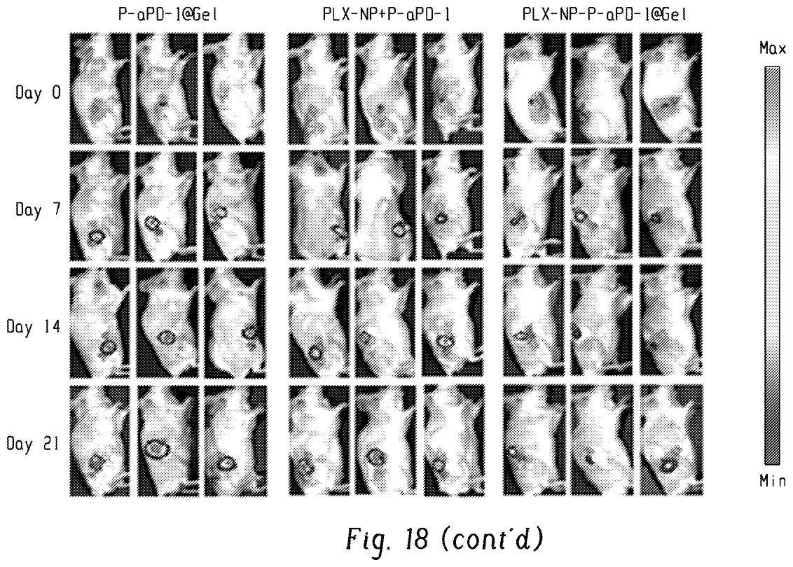

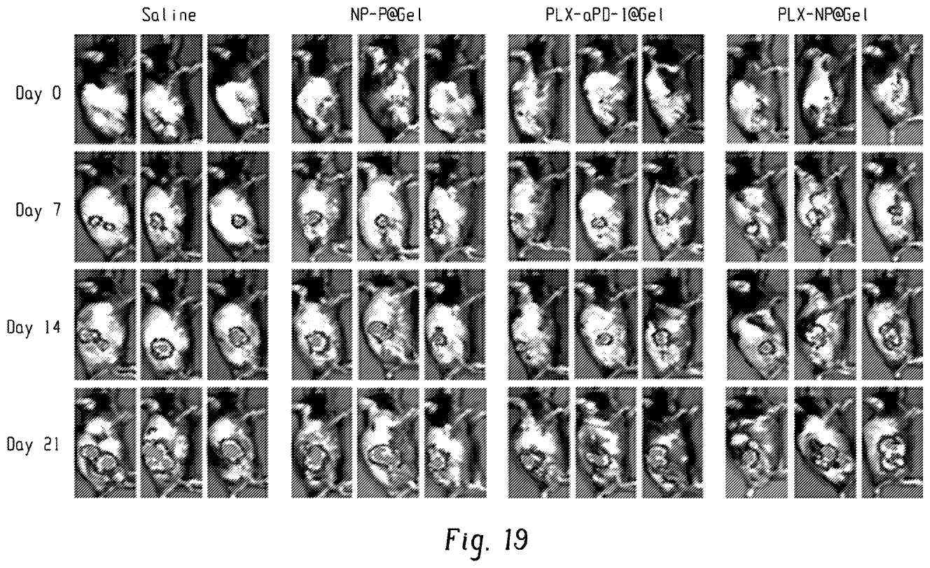

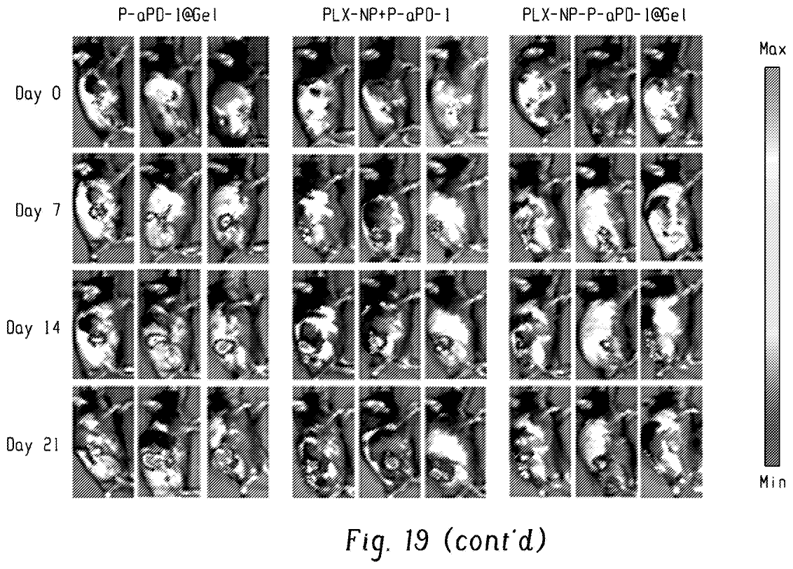

To further evaluate the anti-tumor efficacy of PLX-NP-P-aPD-1@Gel against post-surgery tumor recurrence, the B16F10 melanoma recurrence mouse model was built and treated as previously described. As shown in FIG. 19, after surgery, the bioluminescence intensity of tumor focus was very weak and there was no significant difference among different groups. As shown in FIG. 3D, while the saline group and NP-P@Gel group showed negligible anti-tumor effects, mice treated with PLX-aPD-1@Gel, PLX-NP@Gel, P-aPD-1@Gel, PLX-NP+P-aPD-1, and PLX-NP+P-aPD-1@Gel resulted in varying efficacy in inhibition of tumor growth, evidenced by a sharper increase of bioluminescence signals in the saline and NP-P@Gel groups compared with all other groups. The implantation of PLX-NP@Gel and P-aPD-1@Gel at the surgical cavities displayed moderate tumor growth inhibition effects, indicating the limited therapeutic efficacy of localized treatment of PLX-NP or P-aPD-1 as a monotherapy strategy. While in combination therapeutic groups, mice treated with PLX-aPD-1@Gel and PLX-NP+P-aPD-1 did not display promising anti-tumor effects. However, mice treated with PLX-NP-P-aPD-1@Gel showed the most prominent protection from tumor recurrence, which substantiated the superiority of hydrogel as a local reservoir for sustained and bioresponsive release of PLX and aPD-1. It is worth noting that a single treatment of a low dose of P-aPD1 (0.1 mg/kg) displayed a significant improvement in inhibition of tumor recurrence when integrating with PLX-NP into the hydrogel, highlighting the advantages of convergence of TAMs depletion and immune checkpoint blockade. The more potent anti-tumor activity of PLX-NP-P-aPD-1@Gel was further demonstrated by the prolonged survival time of the mice compared with all other groups (FIG. 3E). Moreover, no significant weight loss of mice was observed in all treatment groups during the time-course of treatment, suggesting negligible potential toxicity against mice (FIG. 3F).

### EXAMPLE 4: PLX-NP-P-APD-1@GEL EFFICIENTLY DEPLETED TAMS AND ENHANCED CD8+ T CELL INFILTRATION

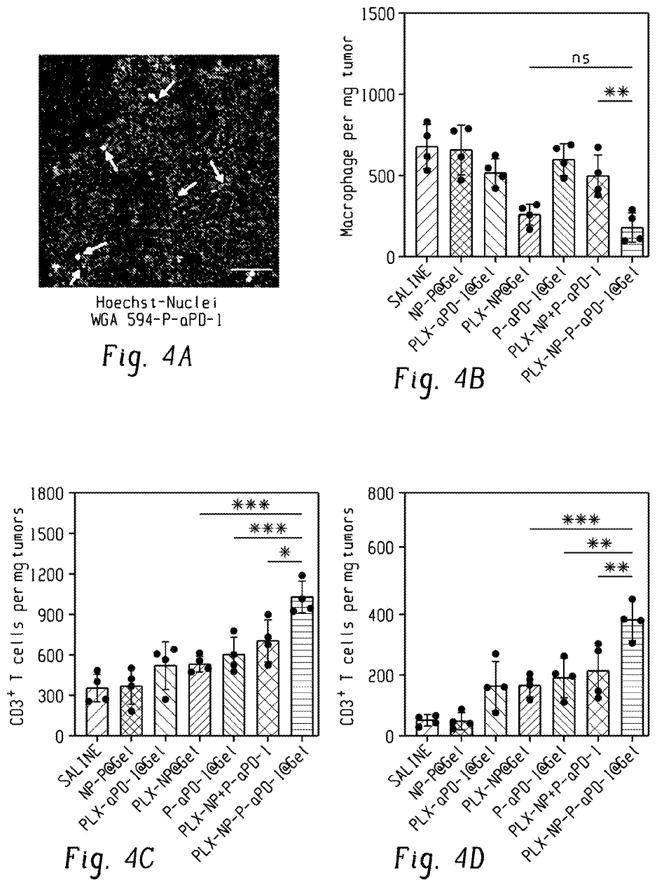

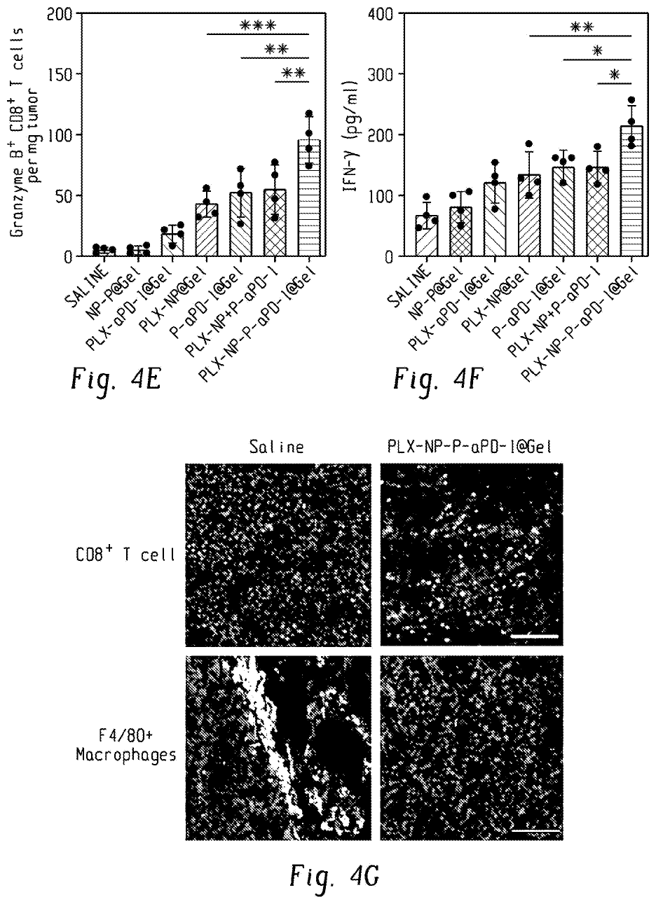

To further investigate the underlying mechanism of the superior anti-tumor efficacy of PLX-NP-P-aPD-1@Gel, the undergoing activation of P-aPD-1 was firstly visualized in the confocal images of tumors treated with WGA 594-labeled P-aPD-1 for 48 hours after surgery (FIG. 4A), as evidenced by the observation of the existence of PMPs in the tumor tissues. The production of PMPs that was attributed to in situ activation of platelets could release aPD-1 antibodies in a bioresponsive manner, further revoking exhausted CD8^{+ T cells for enhanced anti-tumor efficacy. Moreover, the activation of platelets could also help recruit CD}8^{+ T cells by secreting various chemokines and cytokines, including CD}40L26 and RANTES27, strengthening the effects of immune checkpoint blockade treatment. Next, after the various treatments, tumor tissues were collected for flow cytometry analysis. As shown in FIG. 4B, the numbers of macrophages in the tumor tissues implanted with all hydrogels containing PLX-NP formulations were decreased compared with other groups, in which PLX-NP-P-aPD-1@Gel displayed 73.7% deduction of macrophages when compared to the saline group. While there was no significant difference in the density of macrophages between the PLX-NP-P-aPD-1@Gel group and the PLX-NP@Gel group.

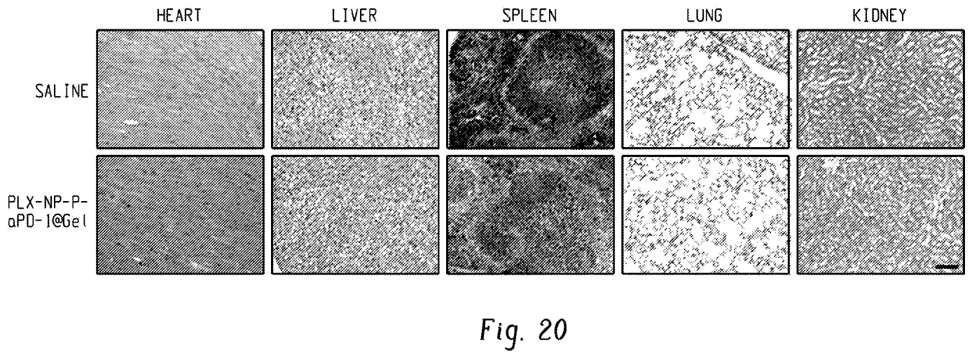

T cells in the tumor tissues were further analyzed to investigate if there was any enhancement of infiltrated T cells after depletion of TAMs. As shown in FIG. 4C, the population of CD3⁺ T cells in the PLX-NP@Gel and P-aPD-1@ gel group was increased by 1.5-fold and 1.7-fold, respectively, when compared with the saline group. Furthermore, a significantly enhanced CD3⁺ T cell infiltration was observed when co-delivering PLX-NP and P-aPD-1 in a hydrogel, as evidenced by 2.8-fold, 1.9-fold, and 1.7-fold increases in the percentage of CD3^{+ T cells compared with saline, PLX-NP@Gel, and P-aPD-}1@Gel groups, respectively. In addition, the significantly increased numbers of CD8⁺ T cells in the tumor tissues treated with all aPD-1 formulations were observed compared with other treatment groups, while the highest density of CD8⁺ T cells in the tumor tissue was achieved in the PLX-NP-P-aPD-1@Gel group, which was 2-fold of that in P-aPD-1@Gel group, highlighting that the TAMs depletion by PLX-NP contributed substantially to the enhanced infiltration of CD8+ T cells (FIG. 4D). Meanwhile, a significant increase in Granzyme B+CD8+ T cells population in the PLX-NP-P-aPD-1@Gel group compared with all other treatment groups, indicating enhanced effector T cell population for anti-tumor effects (FIG. 4E). Furthermore, the IFNγ cytokine level was elevated in the PLX-NP+P-aPD-1@Gel group (FIG. 4F), demonstrating the strengthened immune response. To further demonstrate TAMs depletion and improved CD8⁺ T cells infiltration, immunohistochemistry staining was performed on tumor tissues. As shown in FIG. 4G, a substantial depletion of TAMs and the increased numbers of infiltrated CD8⁺ T cells were observed in the PLX-NP-P-aPD-1@Gel group than that of the saline group. And then, the hematoxylin and eosin (H&E) staining images of the main organs of the mice proved therapeutic safety of the PLX-NP-P-aPD-1@Gel treatment strategy with negligible systemic toxicity compared to saline-treated mice (FIG. 20). Collectively, the results revealed that the TAMs depletion by PLX-NP released from the hydrogel facilitated the infiltration of CD8⁺ T cells by modulating tumor immunosuppressive microenvironment, which was further activated by aPD-1 antibodies released from P-aPD-1, enhancing immunotherapeutic efficacy.

### EXAMPLE 5: IN VIVO ANTI-TUMOR EFFICACY OF THE PLX-NP-P-APD-1@GEL IN 4T1 BREAST TUMOR RECURRENCE AND METASTASIS MODEL

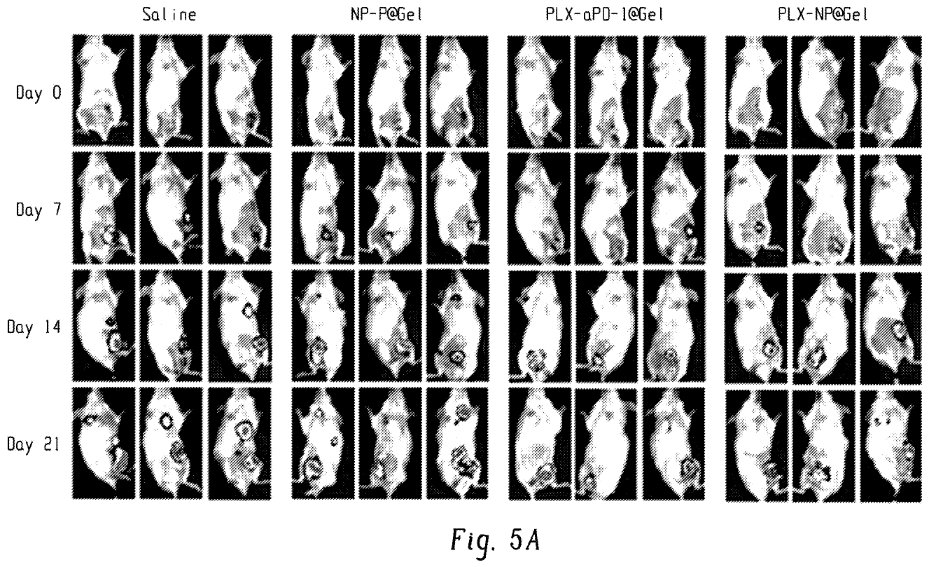

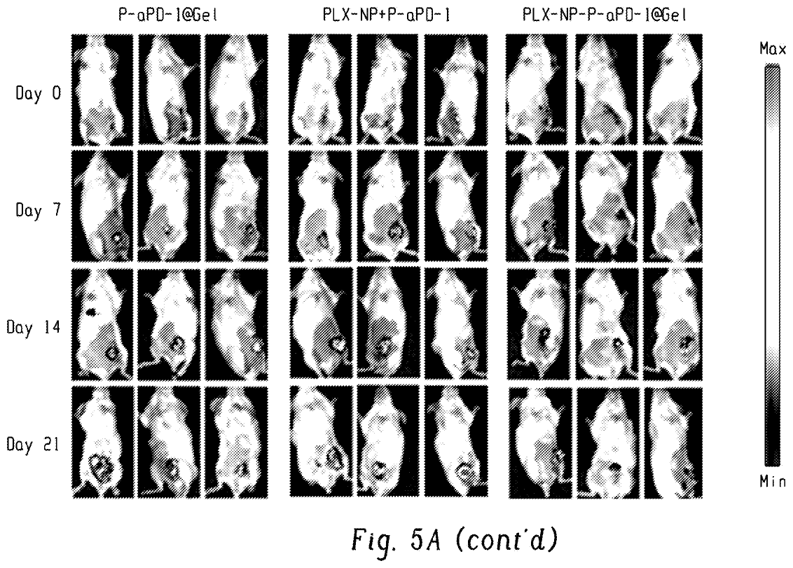

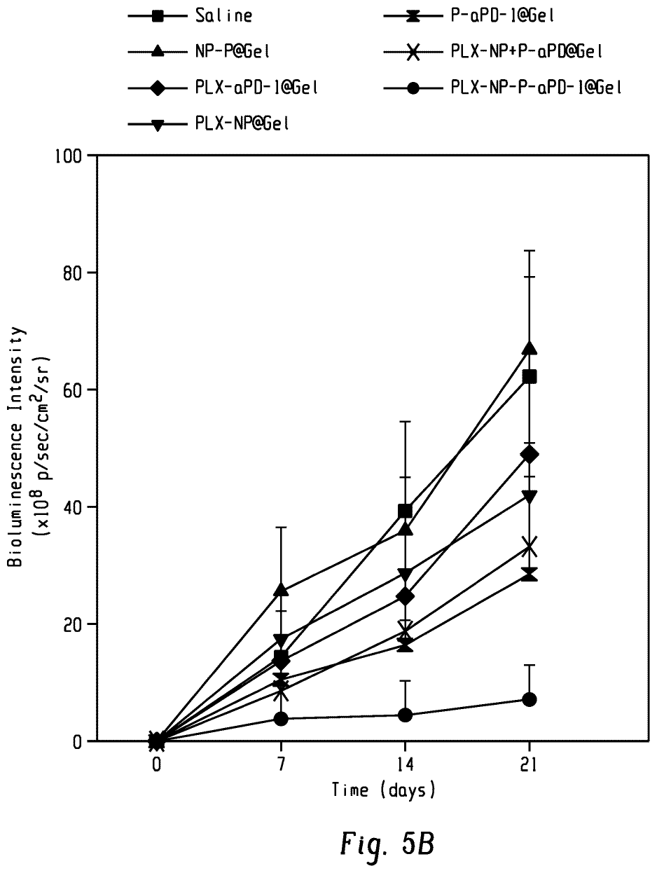

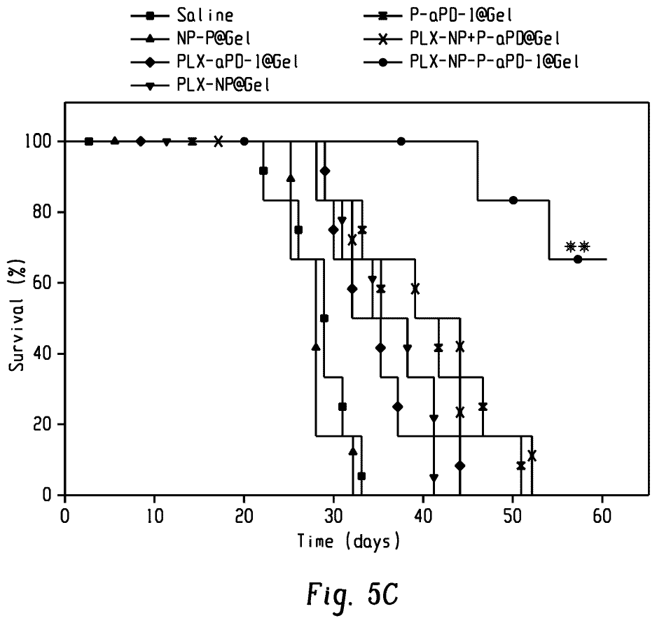

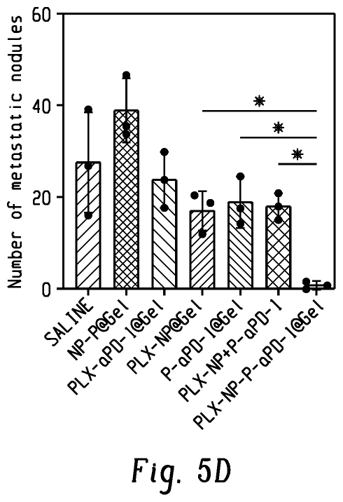

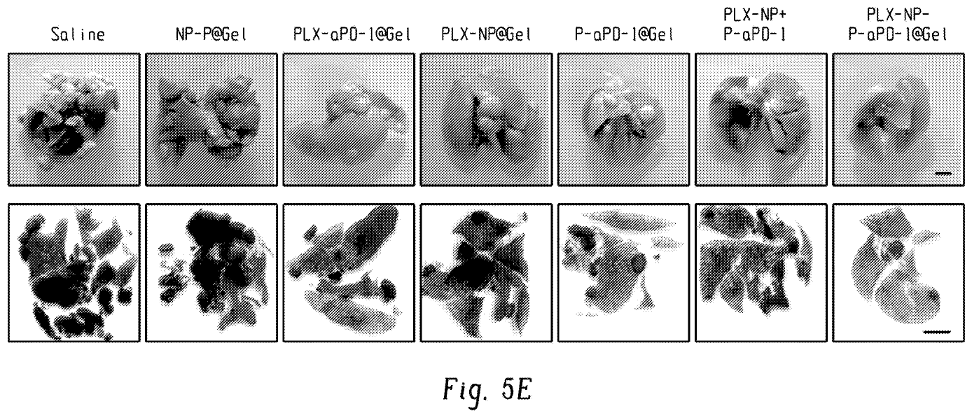

Clinically, triple-negative breast cancer cells are usually very aggressive and metastatic. Therefore, to further demonstrate the immunotherapeutic efficacy of PLX-NP-P-aPD-1@Gel in a metastatic tumor model, the 4T1 breast tumor recurrence mouse model was established and treated with various treatment groups as before. The recurrent tumor growth was monitored by measuring bioluminescence signals from luciferase-tagged 4T1 cells during the time-course of treatment. As shown in FIG. 5A, the surgical bed displayed very weak bioluminescence signals and there is a negligible difference among different groups. According to the results, in the saline group, the bioluminescence signal began to appear in the lung area on day 14, and already became very strong on day 21, indicating that the tumor had recurred from the surgical site and metastasized to the lungs. The NP-P@Gel showed almost no therapeutic effect for both the tumor recurrence and lung metastasis. Notably, the PLX-NP-P-aPD-1@Gel, potently inhibits tumor recurrence and growth, showing significantly better anti-tumor efficacy than the PLX-aPD-1@Gel, PLX-NP@Gel, P-aPD-1@Gel, and PLX-NP+P-aPD-1 (FIG. 5B). Moreover, the mice in the saline group all died within 33 days, while after the treatment of PLX-NP+P-aPD-1, more than 66% of mice survived 60 days, showing a better survival prolongation effect than other treatments (FIG. 5C). In addition, another batch of 4T1 breast tumor recurrence model was established and corresponding treatments were performed to better explore the effects of the treatment strategy for the inhibition of lung metastasis. It was found that the PLX-NP-P-aPD-1@Gel treatment could significantly decrease the number of metastatic nodules on the lung surface compared with other treatment groups (FIG. 5D). Also, from the images and H&E assay of the lungs (FIG. 5E), severe lung metastasis was observed in the saline group and it was found that the PLX-aPD-1@Gel, PLX-NP@Gel, P-aPD-1@Gel, and PLX-NP+P-aPD-1 treatment inhibited related lung metastasis to a certain extent. Encouragingly, the PLX-NP-P-aPD-1@Gel exhibited the most potent inhibition of lung metastasis, which is also an important reason for its superior survival prolonging effect.

### EXAMPLE 6: IN VIVO ANTI-TUMOR EFFICACY OF THE PLX-NP-P-APD-1@GEL IN THE SARCOMA S180 TUMOR MODEL, B16F10 TUMOR RECURRENCE MODEL IN T CELL-DEFICIENT MICE, AND B16F10 DISTANT TUMOR MODEL

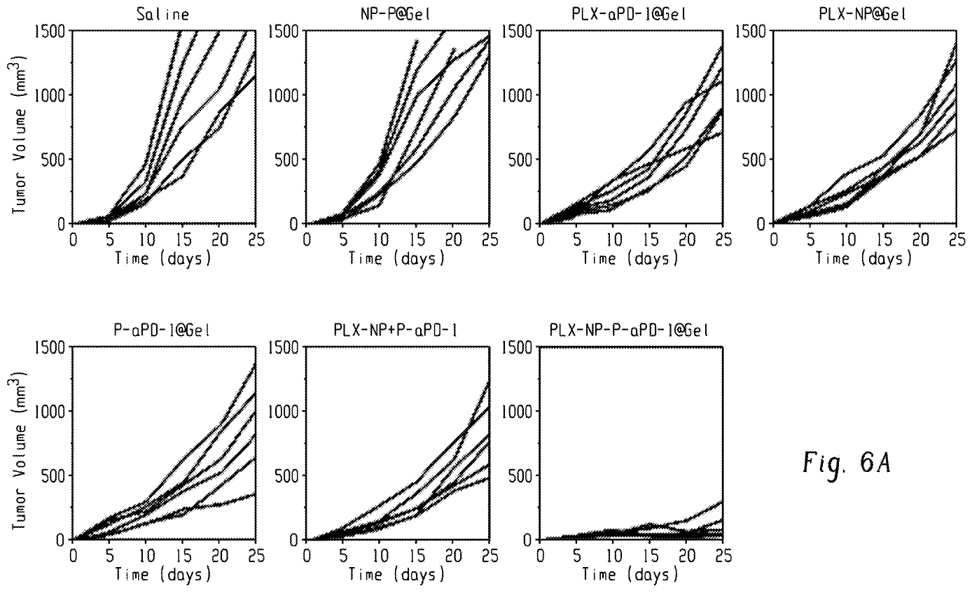

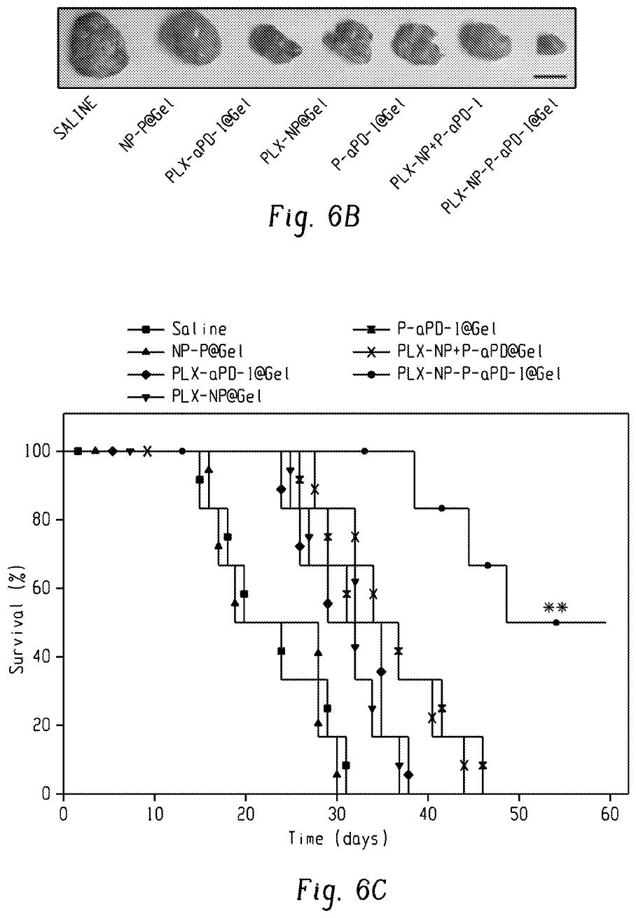

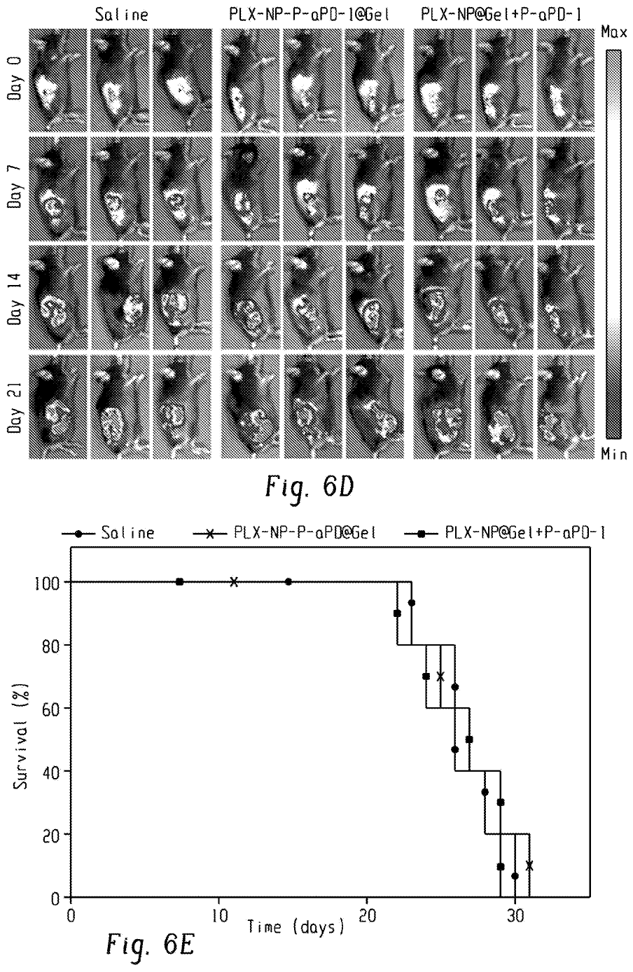

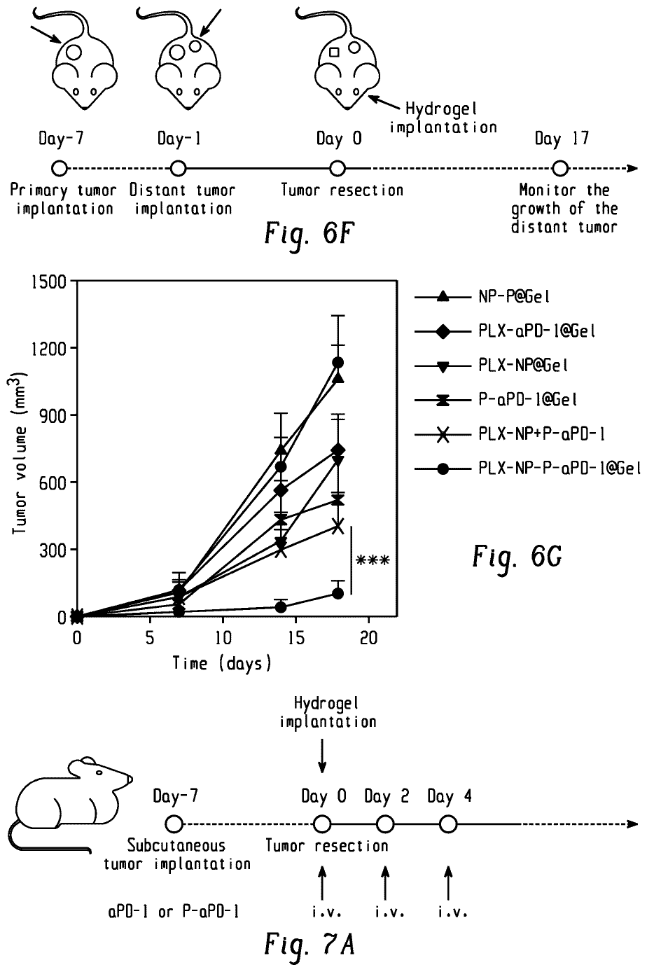

In a clinical situation, the boundary between some tumors and tissues is not clear, such as the sarcoma tumor model, so it is difficult to judge when they are surgically removed. Here, a well-known sarcoma S180 tumor model was established, having rapid growth and proliferation. The recurrence and growth of the tumor were monitored after the surgery and treatment with PLX-NP-P-aPD-1@Gel. The tumors in the saline group recurred and grew fast with the tumor bearing mice all dead in 31 days (FIGS. 6A, 6B). Although the PLX-aPD-1@Gel, PLX-NP@Gel, P-aPD-1@Gel, and PLX-NP+P-aPD-1 treatment showed an inhibition effect on the tumor recurrence and growth, the mice still all died within 46 days. Notably, the PLX-NP-P-aPD-1@Gel treatment strategy significantly inhibited the growth of the recurred tumor and 50% of the mice survived to 60 days (FIG. 6c).

Furthermore, to demonstrate the importance of T cells in the hydrogel based post-surgery immunotherapy, the B16F10 melanoma model was established in T cell-deficient rag-mice, and the treatment efficacy of PLX-NP-P-aPD-1@Gel and PLX-NP@Gel+P-aPD-1 was verified by measuring the bioluminescence signals from luciferase-tagged B16F10 cells. As shown in FIG. 6D, there were no significant differences between each group. The strong bioluminescence signals in the PLX-NP-P-aPD-1@Gel and PLX-NP@Gel+P-aPD-1 groups demonstrated that the hydrogel based post-surgery immunotherapy could not work in the T cell-deficient mice. Moreover, the tumor bearing T cell deficient mice in different treatment groups all died within 31 days further proving that T cells are involved and play an important role in the hydrogel based post-surgery immunotherapy. In order to verify if the localized treatment strategy could activate the whole immune system to inhibit the growth of the distant tumor, a B16F10 distant tumor model was established. Specifically, a primary B16F10 tumor model was first established on the back of the right side of the mouse on day-7 and then a distant tumor model was later established on the left side of the mouse on day-1. On day 0, the primary tumor was resected and the hydrogel delivery systems were implanted into the surgical bed, and the distant tumor volumes were monitored to day 17 (FIG. 6F). According to FIG. 6G, compared with other treatments, the growth of the distant tumor in the PLX-NP-P-aPD-1@Gel was significantly inhibited, demonstrating that the local treatment strategy could activate the whole immune system to inhibit the distant disease.

### EXAMPLE 7: IN VIVO ANTI-TUMOR EFFICACY OF LOCAL IMPLANTATION OF PLX-NP@GEL AND SYSTEMIC INJECTION OF P-APD-1

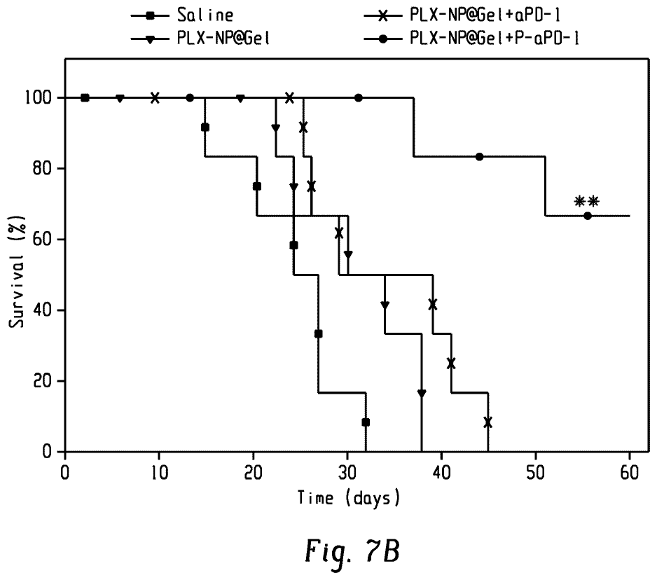

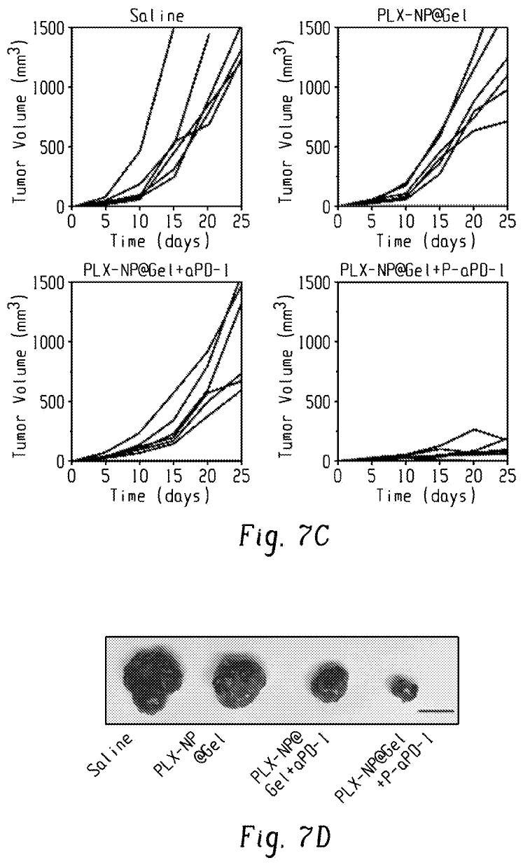

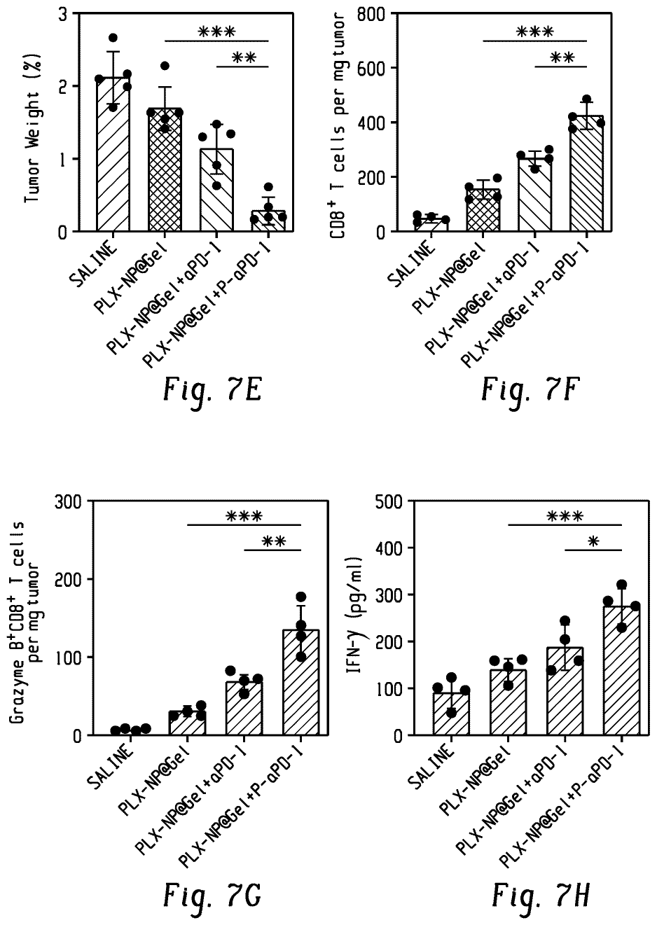

To investigate whether localized implantation of PLX-NP@Gel could enhance the anti-tumor effect of the systemic injection of P-aPD-1, the therapeutic efficacy of PLX-NP@Gel, and systemic administration of P-aPD-1 (designated PLX-NP@Gel+P-aPD-1) was evaluated in the B16F10 tumor recurrence model as mentioned previously. After tumor resection, different treatments were applied, including saline, PLX-NP@Gel, PLX-NP@Gel with systemic injection of free aPD-1 antibodies (PLX-NP@Gel+aPD-1), and PLX-NP@Gel+P-aPD-1 every other day for three times starting from day 0 (FIG. 7A). Notably, the mice in PLX-NP@Gel+P-aPD-1 group showed markedly prolonged survival time compared with the mice in other groups (FIG. 7B), while the mice treated with saline all died in 32 days. Furthermore, as shown in FIG. 7C, PLX-NP@Gel and PLX-NP@Gel+aPD-1 treatments moderately slowed down the growth of recurrent tumors in the mice compared with the saline group but eventually failed to inhibit the tumor growth. In contrast, the tumor recurrence and growth were significantly prevented by PLX-NP@Gel+P-aPD-1 treatment.

The enhanced tumor inhibition of PLX-NP@Gel+P-aPD-1 compared to PLX-NP@Gel+aPD-1 highlighted the critical role of platelets in the in vivo delivery of immune checkpoint inhibitors, which could improve 1) pharmacokinetics of aPD-1 antibodies by prolonging the circulation time (Hu et al, Nature biomedical engineering 2018, 2(11):831-840); 2) accumulation of aPD-1 antibodies at the tumor site by leveraging the homing capability of platelets towards wound site and tumor tissue (Menter et al, Cancer and Metastasis Reviews 2017, 36(2):199-213; Hu et al, Advanced Materials 2015, 27(44):7043-7050). Furthermore, as visualized in FIG. 7D, mice treated with PLX-NP@Gel+P-aPD-1 carried significantly smaller tumors having the lowest tumor weight among all treatment groups (FIG. 7E).

The immune response was further investigated by flow cytometry and the detection of cytokines by ELISA after PLX-NP@Gel+P-aPD-1 treatment. Enhanced CD8⁺ T cells in PLX-NP@Gel and PLX-NP@Gel+aPD-1 groups were quantitatively demonstrated with 3.3-fold and 5.7-fold increases compared with the saline group, respectively (FIG. 7F). In contrast, the PLX-NP@Gel+P-aPD-1 group showed a 1.6-fold greater percentage of CD8⁺ T cells compared with the PLX-NP@Gel+aPD-1 group, which could be attributed to the increased pharmacokinetics and tumor-selective accumulation of aPD-1 mediated by platelets. Moreover, the promoted T cell activation was substantiated by increased Granzyme B⁺ CD8⁺ T cell population in mice treated with PLX-NP@Gel+P-aPD-1compared with all other groups (FIG. 7G). Furthermore, elevated cytokine levels were detected in the PLX-NP@Gel+P-aPD-1 group, as shown by a 3-fold increase in IFNγ (FIG. 7H) and a 4.5-fold increase in TNFα (FIG. 7I) compared with the saline group. Taken together, local TAMs depletion strategy could facilitate both local and systemic treatment efficacy of platelet-mediated delivery of immune checkpoint inhibitors.

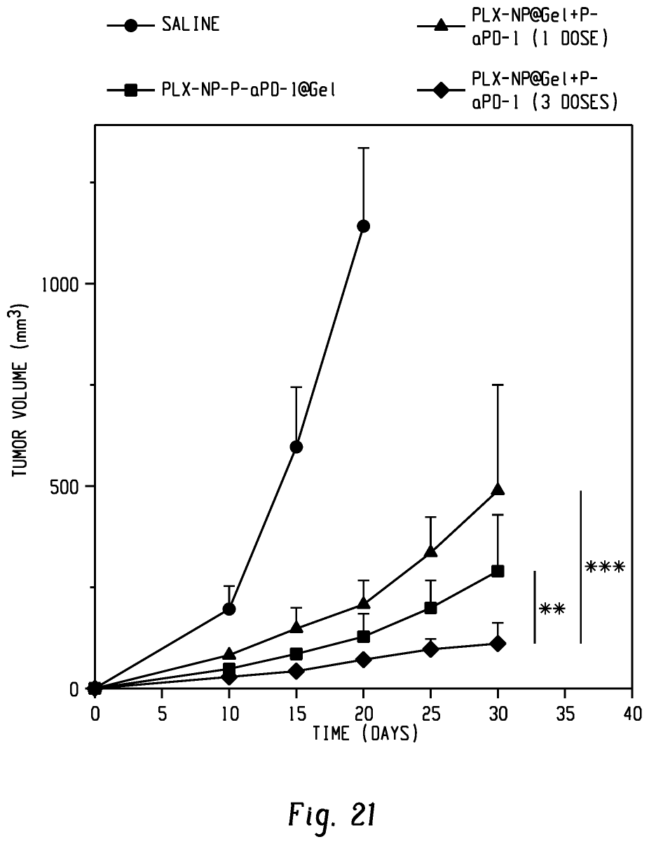

As shown in FIG. 21, multiple treatments with PLX-NP@Gel+aPD-1 had a significantly greater effect on decreasing tumor volume than a single dose.

### CONCLUSIONS

It has been shown that the disclosed hydrogel could act as a local reservoir to sustainedly release PLX-NP and P-aPD-1 for enhanced efficacy of tumor immunotherapy by depleting TAMs to facilitate T cells infiltration and in situ promoting aPD-1 release in a bioresponsive manner for blocking PD-1/PD-L1 pathway to re-activate infiltrated T cells. Furthermore, this local TAMs depletion strategy could be further adapted to enhance the treatment outcomes of systemic platelet-mediated aPD-1 delivery.

Inhibition of the TAMs by blocking CSF1 receptors is a viable method to modulate tumor immunosuppressive microenvironment to facilitate CD8⁺ T cells infiltration, while the durable treatment outcomes are yet to achieve in the clinical partially due to the nonspecific distribution of CSF1 receptor inhibitors that could also deplete the macrophages in the healthy tissues, leading to side effects like edema. Herein, a local delivery strategy embeds PLX-NP into a hydrogel implanted in the post-surgery tumor cavity. The hydrogel can act as a depot for controlled and sustained release of PLX concentrated in the tumor tissue against TAMs, which will minimize the side effects toward normal tissues and augment the depletion efficacy of PLX. Furthermore, previous studies have demonstrated that TAMs could impede the infiltration of CD8⁺ T cells, limiting the treatment efficacy of immune checkpoint blockade strategy. Encouragingly, depletion of TAMs could facilitate the migration of CD8⁺ T cells towards tumor parenchyma by blocking the crosstalk between CD8⁺ T cells and TAMs, promoting anti-tumor immune response. However, the delivery of immune checkpoint inhibitors, especially when administrated systemically, often suffers from a quick clearance, diminishing their therapeutical efficacy. In this study, platelets were employed as carriers for aPD-1 antibodies and embedded them into the hydrogel together with PLX-NP. The sustained release of P-aPD-1 could be controlled by the hydrogel, followed by the presentation of aPD-1 towards T cells facilitated by in situ activation of platelets in the inflammatory environment of the post-surgical tumor site. Additionally, the platelet activation in the inflammatory environment secondary to the tumor surgery could also facilitate the recruitment of immune cells, boosting the anti-tumor immune response.

It was further demonstrated that this local depletion of TAMs through the hydrogel reservoir could also augment the immunotherapy efficacy of systemic injection of P-aPD1, diversifying the administration routes of immune checkpoint inhibitors. To be noted, the systemic free aPD-1 injection did not bring significant therapeutic outcomes, which could be attributed to the low availability of aPD-1 antibodies at the tumor site. While the enhanced treatment efficacy in the combination of systemic P-aPD-1 and PLX-NP@Gel suggested the key role of platelets in promoting the systemic biodistribution of intravenously injected aPD-1 by selectively accumulating at the tumor site, subsequently synergizing with TAMs depletion-mediated modulation of tumor immunosuppressive environment for improved efficacy.

Collectively, it was shown that PLX-NP and P-aPD-1 could be delivered as the combination treatment based on an alginate-based hydrogel localized intratumoral delivery after surgical resection, facilitating the treatment efficacy by leveraging the synergy of TAMs depletion and bioresponsive aPD-1 delivery. Further, the local TAMs elimination approach could also improve the treatment outcomes of systemic aPD-1 delivery.

The use of the terms “a” and “an” and “the” and similar referents (especially in the context of the following claims) are to be construed to cover both the singular and the plural, unless otherwise indicated herein or clearly contradicted by context. The terms first, second, etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values is merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A depot pharmaceutical formulation, comprising
a biocompatible hydrogel, the hydrogel encapsulating
an effective amount of a colony-stimulating factor receptor (CSFR) inhibitor, wherein the CSFR inhibitor blocks CSF receptors on tumor associated macrophages (TAMs); and
an effective amount of a platelet-conjugated anti-immune checkpoint inhibitor (ICI) antibody, wherein the platelet-conjugated anti-ICI antibody activates T-cells.

2. The depot pharmaceutical formulation of claim 1, wherein the biocompatible hydrogel comprises alginate, polyphosphazine, poly(acrylic acid), poly(methacrylic acid), poly(alkylene oxide), poly(vinyl acetate), polyvinylpyrrolidone (PVP), agarose, chitosan, dextran, dextran sulfate, heparan, heparan sulfate, cellulose sulphate, carrageenan, gellan gum, xanthan gum, guar gum, chondroitin sulfate, hyaluronic acid, collagen, gelatin, poly(N-isopropyl acrylamide), or a combination thereof.

3. The depot pharmaceutical formulation of claim 1, wherein the biocompatible hydrogel comprises alginate.

4. The depot pharmaceutical formulation of claim 1, wherein the CSFR inhibitor is encapsulated in a biodegradable sustained release nanoparticle.

5. The depot formulation of claim 4, wherein the biodegradable sustained release nanoparticle is a biodegradable polysaccharide nanoparticle.

6. The depot formulation of claim 5, wherein the polysaccharide nanoparticle is a dextran nanoparticle.

7. The depot formulation of claim 1, wherein the CSFR inhibitor comprises pexidartinib, ilorasertib, masitinib, linifanib, ataxilimab, emactuzumab, cabiralizumab, or a combination thereof.

8. The depot formulation of claim 1, wherein the ICI comprises cemiplimab-rwlc, nivolumab, pembrolizumab, pidilizumab, MEDI-0680, PDR001, REGN2810, and BGB-108, AMP-224, an immunoadhesin, BMS-936559, atezolizumab, YW243.55.S70, MDX-1105, MEDI4736, durvalumab, avelumab, ipilimumab, tremelimumab, BMS-986016, urelumab, TRX518, dacetuzumab, lucatumumab, SEA-CD40, CP-870,893, MED16469, MOXR0916, MSB001078C, or a combination thereof.

9. The depot formulation of claim 1, wherein the anti-ICI antibody comprises an anti-PD-1 antibody.

10. The depot formulation of claim 1, wherein the anti-ICI antibody is conjugated to platelets using a bifunctional linker.

11. The depot formulation of claim 10, wherein the bifunctional linker comprises SMCC (succinimidyl-4-(N-maleimidomethyl)cyclohexane-1-caboxylate); MBS (m-maleimidobenzoyl-N-hydroxysuccinimide ester), sulfo-MBS (m-maleimidobenzoyl-N-hydroxysulfosuccinimide ester), GMBS (N-γ-maleimidobutyryloxysuccinimide ester), sulfo-GMBS (N-γ-Maleimidobutyryloxysulfosuccinimide ester, EMCH (N-(ε-maleimidocaproic acid) hydrazide), EMCS (N-(ε-maleimidocaproyloxy) succinimide ester), sulfo-EMCS N-(ε-maleimidocaproyloxy) sulfo succinimide ester), PMPI (N-(ρ-maleimidophenyl) isocyanate), SIAB (N-succinimidyl(4-iodoacetyl)aminobenzoate), SMPB (succinimidyl 4-(ρ-maleimidophenyl) butyrate), sulfo-SIAB (N-sulfosuccinimidyl(4-iodoacetyl)aminobenzoate), sulfo-SMCC (sulfosuccinimidyl 4-(N-maleimidomethyl) cyclohexane-1-carboxylate), sulfo-SMBP (sulfo succinimidyl 4-(ρ-maleimidophenyl) butyrate), EDC (1-ethyl-3-(3-dimethylaminopropyl) carbodiimide hydrochloride), or MAL-PEGSCM (maleimide PEG succinimidyl carboxymethyl).

12. A method of treating a post-surgical tumor site in a subject, comprising depositing at the post-surgical tumor site an effective amount of the depot formulation of claim 1.

13. The method of claim 12, further comprising systemically administering a systemic formulation comprising platelet-conjugated anti-ICI antibodies, wherein the anti-ICI antibodies in the depot formulation and in the systemic formulation are the same or different.

14. The method of claim 12, wherein the tumor is from bladder, breast, cervix, colon, rectal, endometrial, kidney, oral, liver, lung, melanoma, non-small cell lung cancer, ovarian, pancreatic, prostate, sarcoma, small cell lung cancer, or thyroid.

15. A method of treating a post-surgical tumor site in a subject, comprising
depositing at the post-surgical tumor site an effective amount of a depot pharmaceutical formulation, comprising a biocompatible hydrogel, the hydrogel encapsulating an effective amount of a colony-stimulating factor receptor (CSFR) inhibitor, wherein the CSFR inhibitor blocks CSF receptors on tumor associated macrophages (TAMs); and
systemically administering a systemic formulation comprising a platelet-conjugated anti-immune checkpoint inhibitor (ICI) antibody, wherein the platelet-conjugated anti-ICI antibody activates T-cells.
